(19) United States
(12) Patent Application Publication
(10) Pub. No.: US 2005/0069496 A1
(43) Pub. Date: Mar. 31, 2005

(54) Methods of screening, determining, developing and utilizing negative cross resistance toxins

(76) Inventors: Barry Robert Pittendrigh, West Lafayette, IN (US); Larry Lee Murdock, Lafayette, IN (US); Patrick Joseph Gaffney, Mayfield Heights, OH (US)

Correspondence Address: PATRICK W. RASCHE;ARMSTRONG TEASDALE LLP, ONE METROPOLITAN SQUARE, SUITE 2600, ST. LOUIS, MO, 63102-2740, US

(21) Appl. No.: 10/485,477

(22) PCT Filed: Jul. 30, 2002
(86) PCT No.: PCT/US02/24216

(30) Foreign Application Priority Data
Jul. 30, 2001 (US) 60308790
Aug. 20, 2001 (US) 60313608
Aug. 21, 2001 (US) 60313854
Sep. 14, 2001 (US) 60322227

(51) Int. Cl. A61K 49/00

(52) U.S. Cl. 424/9.2

(57) ABSTRACT

A method is provided for evaluating the efficacy of a molecule against a target population, the target population, including a strain resistant to a first toxin. The method comprising determining a susceptible strain in the target population. The susceptible strain being susceptible to the first toxin. The method further comprising selecting for a resistant strain in the target population. The resistant strain being resistant to the first toxin. The method further comprising evaluating the efficacy of the resistant strain with a plurality of molecules to determine a second toxin that is more toxic to the resistant strain than to the susceptible strain. Wherein the target population is at least one of an insect, a fungi, a plant, and a nematode. Methods and systems are provided for determining and developing negative cross resistance factors. The method includes evolving a strengthened NCR toxin from an initial NCR toxin by obtaining an initial NCR compound, selectively increasing the toxicity of the NCR compound, and testing the evolved compound to determine if the evolved compound is a stronger NCR compound than the initial NCR toxin. Methods and systems are also provided for deployment and commercial use of NCR toxins.

This application claims the combined and separate benefit of priority of U.S. provisional application 60/308,790 filed Jul. 30, 2001; priority of U.S. provisional application 60/313,608 filed Aug. 20, 2001; priority of U.S. provisional application 60/313,854 filed Aug. 21, 2001 and priority of U.S. provisional application 60/322,227 filed Sep. 14, 2001; each and all of which are incorporated herein in their entirety by reference.

A portion of the disclosure of this patent document contains material that is subject to copyright protection. The copyright owner has no objection to the facsimile reproduction by anyone of the patent document or the patent disclosure, as it appears in the Patent and Trademark office patent file and records but reserves all copyrights whatsoever.

### BACKGROUND OF THE INVENTION

This invention relates generally to negative cross resistance, and more particularly, to methods and systems for evaluating, determining, developing, and utilizing negative cross resistance to extend and increase the efficacy of molecules to kill unwanted resistant organisms.

Two of the most important scientific events of the twentieth century are the green revolution and the development of pesticides and antibiotics. The green revolution, with the large-scale use of pesticides, i.e., insecticides, herbicides, and fungicides, brought about dramatic increases in quantity and quality of food for an ever-growing human population, allowing for reliable food sources for billions of people on this planet. In addition, antibiotics dramatically reduced the mortality rates of the human population to bacterial diseases, virtually wiping out some bacterial epidemics. However, (pesticide and antibiotic) resistance has evolved due to the large scale use of the pesticides and antibiotics.

Although efforts have been made to slow the development of such resistance, the evolution of resistance is generally considered inevitable. Once widespread resistance develops which is unfortunately inheritable, the chemical (or chemical-class) that resistance has developed against is typically abandoned thus causing loss of capability of using such pesticides and antibiotics, the incurring of significant expense to develop new ones and needless interruption in field and management of land practices. The subsequent focus in the research and industrial community is to identify novel pesticides and antibiotics with different modes of action, where positive cross-resistance to previously used biocides does not occur. One alternative to the seemingly endless treadmill practice of discarding old and existing compounds and continually seeking new compounds is the development of negative cross-resistance strategies to control organisms containing the resistance allele.

Negative cross-resistance (NCR) as a strategy for pesticide resistance management encompasses a scenario where organisms tolerant to one compound are highly sensitive to another compound and vice versa. For example, if a pest population is treated with a toxin such as pesticide ‘A’, the number of pests carrying alleles resistant to pesticide ‘A’ will increase in frequency. After numerous generations, pests carrying the resistance allele will comprise the majority of the population. At this time a second toxin that preferentially kills those pests tolerant to the first toxin is used to treat the pest population. Use of the second toxin changes the frequency of the alleles such that the first toxin can again be used to control the pests' population for one to several generations. By alternately deploying the two toxins, a NCR strategy is used to maintain effective control of the pest while ‘managing’ the resistance alleles in the pest population.

Negative cross-resistance has been shown to occur with a wide variety of organisms. It has been observed with pesticides used to control German cockroaches, diamondback moths, mosquitoes, spider mites, and horn flies. Additionally, this phenomenon has been observed in weeds resistant to herbicides, fungal strains resistant to fungicides, and in bacteria resistant to antibiotics. Negative cross-resistance has been associated, in some instances, with a single amino acid change in the targeted allele. For example, negative cross-resistance in Ustilago maydis between the fungicides benzimidazole and diethofencarb was reportedly due to a mutation at a single locus.

Even where practically applicable toxin pairs exist, there is typically little understanding of how such toxins affect the frequency of resistance alleles. Although negative cross-resistance occurs across a wide array of toxins and organisms, the use of a pair of second generation pesticides in a NCR strategy has rarely been commercially applied. One of the few practically applicable NCR strategies is with N-methylcarbamate and N-propylcarbamate to green rice leafhopper Nephotittix cincticeps Uhler, whose populations contain both mutant and wild-type acetylcholinesterases. Use of N-propylcarbamate on the green rice leafhopper population resulted in a population more susceptible to N-methylcarbamate and vice versa. Researchers have been able to shift the resistance level back and forth by alternating between the two aforementioned carbamates.

One reason put forth for such little commercial applicability is because much of the discovery effort in industrial entomology has focused on finding toxins with novel modes of action and improving the efficacy and spectrum of action of already discovered toxins.

One of the limitations to developing NCR toxins as with any type of biocide is that determining highly effective toxins requires screening a large number of test compounds. A methodology to reduce the numbers of lead toxins that need to be creened will greatly increase the efficiency of development of practical NCR toxins.

Another limitation to developing NCR toxins is that there is currently no use for NCR toxins that will cause only moderate mortality rates in the homozygous and heterozygous resistant organisms. A commercial use for moderately toxic NCR factors greatly increases the need for the development of practical NCR toxins.

Another limitation to developing NCR toxins is that even if moderately effective NCR toxins are determined through screening and there is currently no way to evolve these toxins into highly effective NCR toxins. A methodology for evolving moderately effective NCR toxins into highly effective NCR toxins will greatly increase the efficiency of developing practical NCR toxins.

Another limitation to developing NCR toxins is that currently no business model provides a significant market advantage to those companies that develop these toxins for use with a biocide that has been previously developed.

### BRIEF SUMMARY OF THE INVENTION

In one aspect of the invention, a method is provided for evaluating the efficacy of a molecule against a target population. The target population includes a bacterial strain resistant to a first toxin. The method comprises determining a susceptible bacterial strain, the susceptible strain being susceptible to the first toxin. The method further comprises selecting for the resistant strain, the resistant strain being resistant to the first toxin. The method further comprises evaluating the efficacy of the resistant strain with a plurality of molecules to determine a second toxin that is more toxic to the resistant strain than to the susceptible strain, wherein the resistant and susceptible bacterial strains coexist in the target population.

In another aspect of the invention, a method is provided for controlling a host species that includes a bacteria existing in a symbiotic relationship with the host species. The method comprises determining a susceptible strain of the bacteria that is susceptible to a first toxin, determining a resistant strain of the bacteria that is resistant to the first toxin, determining a second toxin that is more toxic to the resistant strain than to the susceptible strain, and applying the first toxin and the second toxin to the host species such that the host species is adversely impacted.

In another aspect of the invention, a method is provided for generating a resistant organism to be used in developing NCR toxins. The method comprising creating genomic changes in the organism to create a resistance trait, wherein the resistant organism is generated for the purpose of developing NCR toxins.

In another aspect of the invention, a method is provided for screening for negative cross resistance with respect to a target allele. The method comprising evaluating whether a first toxin that was originally effective against a susceptible line of organism is effective against a susceptible line of organism, and if the toxin is no longer effective against the susceptible line of organism, evaluating whether the toxin is effective against a resistant line of organism.

In another aspect of the invention, a method is provided for evaluating the efficacy of a molecule against a target population, the target population including a strain resistant to a first toxin. The method comprises determining a susceptible strain in the target population, the susceptible strain being susceptible to the first toxin. The method further comprises selecting for the resistant strain in the target population, the resistant strain being resistant to the first toxin. The method further comprises evaluating the efficacy of the resistant strain with a plurality of molecules to determine a second toxin that is more toxic to the resistant strain than to the susceptible strain. In one embodiment, only the resistant strains are evaluated, wherein the target population is at least one of a fungi, a plant, and a nematode.

In another aspect of the invention, a method is provided for testing for negative cross resistance in a target population, the method comprising determining a susceptible strain (S/S) in the target population, the susceptible strain (S/S) susceptible to a first toxin. The method further comprises selecting for a resistant strain (R/R) in the target population, the resistant strain (R/R) resistant to the first toxin. The method further comprising evaluating the efficacy of the resistant strain (R/R) with between about 10 and about 10⁹ molecules to determine a second toxin that is more toxic to the resistant strain (R/R) than to the susceptible strain (S/S), wherein the target population is at least one of a fungi, a plant, and a nematode.

In another aspect of the invention, a method is provided for using a first negative cross-resistance (NCR) toxin and a second NCR toxin against a pest population in a refuge to selectively kill heterozygotes and homozygotes carrying resistance alleles to the first NCR toxin, wherein the first NCR toxin is used in a main field and the second NCR toxin is used in the refuge. The current refuge strategy involves a high dose approach to controlling the pest insects. By high dose is meant an expression level of the toxin in the transgenic plant that will kill 90% or greater of the homozygous or heterozygous susceptible insects.

In another aspect of the invention, a method is provided for using a first negative cross-resistance (NCR) toxin and a second NCR toxin against a pest population in a refuge to selectively kill heterozygotes that carry resistance alleles to the first NCR toxin, which is used in a main field. The “main field” includes the region or locus where the transgenic plants containing the initial toxin are located. In a passive refuge strategy the main (Agricultural) field is where the transgenic plants are located and the refuge contains the non-transgenic plants. In the active refuge the main field is where the plants containing the initial transgenic pesticide are located and the refuge is where the plants containing or being treated with the NCR product are located. In one illustrative example, the main field has transgenic maize, which expresses a Bacillus thuringiensis insecticidal toxin.

In another aspect of the invention, a method is provided for evaluating the efficacy of a molecule against a target population, the target population including a strain resistant to a Bacillus thuringiensis insecticidal toxin (Bt). The method comprises determining a susceptible strain susceptible to a Bacillus thuringiensis insecticidal toxin, selecting for the resistant strain, the resistant strain being resistant to the Bacillus thuringiensis insecticidal toxin, and evaluating the efficacy of the resistant strain with a plurality of molecules to determine a second toxin that is more toxic to the resistant strain than to the susceptible strain.

In another aspect of the invention, a method is provided for evolving NCR toxins from weak toxins to strong toxins. The evolution can occur either with a known NCR compound or with newly determined or identified NCR compounds. The evolution can occur with weak NCR compounds, moderate NCR compounds, and strong NCR compounds. In one embodiment, the evolution-occurs utilizing phage-display technology.

In another aspect of the invention, a method is provided for use of NCR toxins in the commercial market place for companies to add value to another product which is the first toxin.

### BRIEF DESCRIPTION OF THE DRAWINGS

FIG. 1 is a graphic illustration of concurrent use of two toxins that are negative cross resistance factors on a target population where both toxins cause equal mortality in homozygous wild-type and mutant insects, and the heterozygotes are unaffected by the combination. The homozygous mortality rates are given for both the SS and RR groups. The alleles go to 50% frequency regardless of the selection pressure on the two homozygous groups (RR and SS).

FIG. 2 is a graphic illustration of concurrent use of two toxins that are negative cross resistance factors on a target population where the two toxins cause equal homozygous mortality rates and the heterozygotes have higher mortality rates than the homozygotes. The higher the heterozygote mortality, the lower the frequency of the resistance allele in the population.

FIG. 3 is a graphic illustration of concurrent use of two toxins that are negative cross resistance factors on a target population where the first toxin causes a 99% mortality rate to the homozygous wild-type insects, the second toxin causes a 70% mortality rate to the homozygous resistant insects, and 70% of the heterozygous target population are killed. When the R allele becomes common in the population (e.g. 50%/o) then the insect population increases dramatically.

FIG. 4 is a graphic illustration of concurrent use of two toxins that are negative cross resistance factors on a target population where both toxins cause a 70% mortality rate in the homozygous wild-type and mutant insect population, and the heterozygotes are either unaffected by the combination or they are much more susceptible. Where 99% of the heterozygous insects are killed, the frequency of resistance goes to 50% rapidly. Where 1% of the heterozygous insects are killed, the frequency of resistance remains rare in the insect population.

FIG. 5 illustrates the effect of starting allelic frequency on the fate of the resistance allele in the presence of two negative cross resistance factors that kill both homozygous lines (susceptible and resistant) at a 50% level and the heterozygotes at a 70% level. If the resistance allele is less than 50% in the population the use of such NCR toxins will result in the resistance allele becoming very rare.

FIG. 6 illustrates a method of screening for compounds to be deployed commercially as NCR factors.

FIG. 6A is a graphic illustration of a conceptual example of how NCR toxins for microorganisms are identified. Persistant and susceptible microorganisms are both placed in an agar plate beside each other for example. The test chemical is jolted on both of the microorganisms. Those compounds that kill the resistant ime, but not the susceptible ime are identified as potential NCR compounds. The assigns shown in FIG. 6A can be done in separate time and for the resist cut and susceptible microorganisms.

FIG. 7 is a Northern blot of a Canton-S (Can-S), DDT-resistant line with no exposure to DDT (Rst(2)DDT-Wisconsin), and a DDT-resistant fly line with exposure to 20 μg /vial of DDT for 24 hours (Rst(2)DDT-Wisconsin-20 μg of DDT). On the left hand site of the figure the blot for the actin control is given and on the right hand side the blot for the CYP6G1 gene is given.

FIG. 8 illustrates a passive refuge model in which the refuge does not include any toxin. The toxin, including but not limited to a Bt toxin, is planted in the main field. There are no transgenic plants in the refuge.

FIG. 9 illustrates an active-refuge model that employs the use of negative cross-resistance (NCR) toxins in the refuge. The toxin, including but not limited to a Bt toxin, is planted in the main field. There NCR toxin is delivered in the refuge.

FIG. 10 illustrates that if NCR toxin is only effective in killing the RR, but not the RS, insects, there is no delay in resistance as compared to the passive refuge strategy. The main field to refuge ratios are given as 1:1, 4:1, 24:1, and 99:1. The mortality rates were high for RR in the field but the RS were not effected by the NCR toxin.

FIG. 11 illustrates that if NCR toxin is effective in killing RS and RR insects, then the active-refuge can dramatically delay the development of a major resistance allele in the insect population. The main field to refuge ratios are given as 1:1, 5:1, and 99:1. In a large active refuge (e.g. 1:1 ratio) the NCR toxin does not have to be very effective against the heterozygous (RS) individuals in order to keep resistance out of the insect population. The NCR toxins must cause high mortality against the heterozygous (RS) individuals.

FIG. 12 illustrates that if a NCR toxin is moderately effective in killing RS and RR insects, then the active-refuge can dramatically delay the development of a major resistance allele in the insect population. The NCR toxins only need to cause moderate levels of mortality in RS and RR insects in order for the active-refuge to keep the resistance allele at low frequency.

FIG. 13 illustrates a method of screening for a dually resistant allele.

FIG. 14 illustrates the effect of the (i) starting allelic frequency (mutation rate and numbers of possible alleles that can confer resistance to the toxins) and (ii) the refuge size on the rate at which resistance enters the population in the presence of a pair to two NCR toxins.

FIG. 14 A illustrates a pCasPer vector useful in making Drosophila melanogaster transgenic for a given gene or an allele of a given gene or several genes or alleles.

FIG. 15 illustrates an alternative screening method with respect to NCR toxins.

FIG. 16 illustrates a method of providing management decisions for the development of NCR factors.

FIG. 17 is a graphical illustration of an application of deltamethrin, DDT, and a combination of deltamethrin and DDT at the same time to para^{tsl} flies, Canton-S flies, and heterozygotes.

FIG. 18 illustrates amino sequences of soluble and phage display recombinant soyacystatin proteins.

FIG. 19 illustrates that the differential papain inhibitory activity of soyacystatins is analogous between phage-displayed and soluble recombinant proteins. The pre-activated papain was initially incubated with N-benzoyl-DL-arginine-betanaphthylamide to establish 100% activity and each given point in the graph is the average of five replicates. (A) Are the phage particles and (B) are the soluble recombinant proteins (Kiowa et al. 1998).

FIG. 20 illustrates a representative experiment of biopanning selection of phage-displayed soyacystatins. Each well in the microtiter plate was initially coated with a solution of PBS with (+) or (−) 30 μg/ml of papain. The phage particles were then purified from those bacteria which were harboring pBluescript, pSSLM⁻³⁻⁹³ (scLM⁻³⁻⁹³) or pSSNM⁸⁻¹⁰³(ScNM⁸⁻¹⁰³) and the binding reaction was performed in the presence (+) or absence (−) of a competitor (chicken cystatin). Then, the number of bound phage particles was later determined as XL-1 blueCFU and was also normalized to the CFU of the competitor (papain) treatment for each of the phage preparations. The given bars represent the standard deviation (Kiowa et al. 1998).

FIG. 21 illustrates that Papain biopanning selection differentially enriched for scNM⁸⁻¹⁰³ phage particles to a substantially greater extent than for scLM⁻³⁻⁹³ phage particles. Details of this experiment are given in Koiwa et al. (1998). This experiment shows conceptually that biopanning allow for selection of toxins that preferentially interact with an insect target site.

FIG. 22 illustrates that the scNM⁸⁻¹⁰³ protein substantially inhibited cowpea weevil growth and development and resulted in high insect mortality, whereas the scLM⁻³⁻⁹³ protein was relatively inactive. An individual cowpea weevil egg was placed onto an artificial seed containing no (control) or increasing concentrations of soyacystatins. Ten seeds were used per treatment. The within seed developmental time (the upper panel) was determined as a period between when the eggs of the insect hatched and when the adults emerged from the seeds. The bars indicate the standard deviation around the mean. In the lower aned the insect mortality is given. This was calculated as a percentage of total eggs, from the number of the insects that failed to emerge from the artificial seeds (Koiwa et al. 1998)

FIG. 23 illustrates the identification of proteins that have affinity to a target, in this case FVIIa and illustrates sequences of Phage Clones Selected from Na\dot{i}ve and Partially Randomized Libraries for Binding to TF-FVIIa. (Dennis et al. 2001). The amino acids in the boxes show regions where this selection methodology showed these amino acids to be important for binding to the target.

FIG. 24 illustrates the inhibition of Factor X Activation and A-183-b binding by A-Series peptides. (Dennis et al. 2001). This Shows that the residues defined by hard selection would result in molecules that were effective against a target site.

FIG. 25 illustrates sequence preferences using full randomization. (Dennis et al. 2001). The amino acids shown to increase interactions through hard selection were held constant and are shown in the boxes in the figure and underlined in the following: X₃WEVXCWXWEXCX₆. At the X positions all 20 amino acids were substituted. The amino acids that were identified at the given randomized positions are plotted as a function of the preference for interactions with the target. The preference for any specific amino acid is given in the number of standard deviation units (σ) away from a random chance occurrence. This shows that soft selection can further increase the efficacy of a protein against its target.

FIG. 26 illustrates the effect of A-series peptides on the prothrombin time (PT) and activated partial thromboplastin time (APTT) in human plasma. (Dennis et al. 2001). In the upper panel the fold prolongation of the clotting times upon initiation by TF and Ca²⁺ is given. TF is the specific target site that the proteins have been selected to interact with in solution. In the lower panel initiation by actin FS in the APTT assay. The polypeptides had not been selected to interact with actin FS. In both the upper and lower panels these are plotted against concentrations of the selected peptides: A-183 (closed circle), A-100 (closed box), and A-100-Z (open box). The upper panel shows that polypeptides that have been developed by selection can be more effective in prolonging clotting time. In the lower panel neither A-183 nor A-100-Z prolonged clotting times. This showed that A-183 and A-100-Z were specifically interacting with the TF, which they had been selected with as a target site.

FIG. 27 illustrates a flow chart for evolution of a NCR toxin.

FIG. 28 illustrates a method of switching back and forth the sale or delivery in the environment of an initial pesticide and a NCR toxin. The toxins can be applied to, for example but not limited to, an agricultural field to control a pest organism, such as but not limited to an insect.

FIG. 29 illustrates a method of switching back and forth the sale or delivery in the environment of an initial pesticide and a NCR toxin. The toxins can be applied to, for example but not limited to, an agricultural field to control a pest organism, such as but not limited to an insect population.

FIG. 30 illustrates a method of sale or delivery in the environment of an initial pesticide and a NCR toxin. The toxins can be applied to, for example but not limited to, an agricultural field to control a pest organism, such as but not limited to an insect population.

### DETAILED DESCRIPTION OF THE INVENTION

Exemplary embodiments of systems and methods that facilitate evaluating, determining, developing, and utilizing negative cross resistance (NCR) toxins to extend the efficacy of molecules to kill unwanted organisms are described below. The systems and methods facilitate, and are useful, for example, for evaluating the efficacy of molecules against a target population including a strain resistant to a first toxin, utilizing NCR in an active refuge system, evaluating whether a third allele will arise as a result of using a pair of NCR toxins that confers resistance to both toxins, evaluating the efficacy of molecules for NCR in a target population, determining NCR toxins and the strength of such NCR toxins, developing the determined NCR toxins into strengthened NCR toxins, and deploying the strengthened NCR toxin into the field. Although the methods are often described in terms of an entire process, it should be understood that each method, and step in each method, can be used alone, or in combination with any of the other methods and steps described hereinafter.

As used hereinafter, the term molecules includes, but is not limited to, natural molecule(s), synthetic molecule(s), chemicals, compounds, biotechnical species, and biotechnical moieties.

As used herein the term “company” includes any human entity including without limitation an individual person acting on his/her own or as well as a person or persons acting as a company, corporation, corporate entity, partnership, association, business group and multiple persons and the like.

As used herein the term “biotechnical” includes organisms or subcomponents of an organism that have been developed or altered using biotechnology. In one example, a subcomponent of an organism includes, but is not limited to, a toxin that the organism produces. In addition, evaluating the efficacy includes testing, screening, and determining the ability of the toxin to cause mortality in a given population of an organism. Efficacy includes the power or ability to produce an effect. An illustrative effect is increased mortality in the population of a given population of an organism.

As used hereinafter, the terms “delivered” and “deliver” includes applied to, applied, used, applied on, contacted with, sprayed on, a placed in effective contact with a desired target such as a pest.

Further, the term “target population” includes a pest population which includes any living organism growing where it is unwanted, including, but not limited to, at least one of a weed population, a bacterial population, an insect population, a fungus population, a virus population, and a population of disease contributing organisms living in a body of a higher order organism, such as an insect or a mammal.

A strain, for example, a pest strain, includes at least one of a genotype, a phenotype, a genotype and a phenotype, a group of genotypes, a group of phenotypes, and a group of genotypes and phenotypes, that display a response to a toxin in terms of a life history parameter. A life history parameter is defined in terms of mortality rates (Lethal time 50 (LT50) or lethal dose 50 (LD50)) or developmental time or other terms. In one embodiment, a strain contains at least one genotype or phenotype that is resistant or susceptible. Resistance and susceptibility are relative terms and are defined in relation to each other (resistance ratio). Resistance is defined as a genotype or phenotype that requires high rates of application of a chemical, compound, an organism, a toxin, or molecules thereto or thereon to achieve high mortality rates or in which high rates of application of the chemicals do not result in high mortality rates (high efficacy) in the resistant strain. High rates, in practical terms include levels of application of the toxin above the acceptable label rate as defined by regulatory agencies.

In one embodiment, a resistance ratio is the LD50 of resistant pests (strain 1) divided by the LD50 of susceptible pests (strain 2). For example:
Resistance Ratio=LD50 of strain 1 divided by LD50 of strain 2

The resistance ratio can be provided as a number (e.g. 2) or as a numerical ratio 2:1. In one embodiment, a resistance ratio indicating resistance is defined, between strain 1 and strain 2 as 1.5:1 or greater (e.g. 10,000,000:1 ratio). This ratio covers a broad range of toxic ratios. For example, a 1:10,000,000 ratio indicates that the resistant pest could contact crystals of the toxin without being adversely impacted (or damage).

Table 1 illustrates the concurrent use of two toxins to minimize both the frequency of resistance alleles as well as the size of a target pest population, for example, an insect population. Table 1 shows the impact of the toxicity of the second toxin on both the change in allelic frequency of the resistance allele and the effective control of the pest population. In the example illustrated in Table 1, the first toxin causes a 99% mortality rate in the homozygous wild-type pests.

TABLE 1

| | | 50% Allelic Frequency | |
|---|---|---|---|
| Mortality Rate of | | Days to | |
| Homozygous | | (50% Allelic | Mean Population |
| Resistant | Heterozygous | Frequency) | Size |
| 1 | 1 | 100.5 | 9368 |
| | 10 | 100.5 | 7446 |
| | 50 | 109.0 | 2342 |
| | 90 | 138.0 | 328 |
| | 99 | 260.5 | 99169 |
| 50 | 50 | 117 | 2569 |
| | 70 | 123 | 1178 |
| | 90 | 152 | 171 |
| | 99 | 275 | 3287 |
| 90 | 90 | 191.5 | 81 |
| | 99 | 333 | 2 |
| 99 | 99 | Population | |
| | | Crashed | |

If both toxins do not have similar levels of toxicity on the homozygous lines they impact, the allele susceptible to the least toxic compound will increase in frequency. As the allele susceptible to the least toxic compound becomes more common, the toxin-pair becomes less effective in controlling the pest population. In addition, both toxins must kill the heterozygous and homozygous pests at a high rate in order to keep the pest population below economic or a fixed or set thresholds.

Table 2 illustrates the concurrent use of two toxins where the homozygous mortality rates are equal for the two compounds and the heterozygotes have either lower or higher mortality rates than the homozygotes.

TABLE 2

| Mortality Rate of Homozygous | Heterozygote | Fate of Resistance |
|---|---|---|
| Resistant/Susceptible Genotypes | Mortality | Allele at the End of Test |
| 99%/99% | 1% | Asymptotic to 50% |
| | 90% | Asymptotic to 50% |
| | 99% | Population Crashed |
| 50%/50% | 5% | 27.3% |
| | 10% | 20.9% |
| | 50% | 0.14%† |
| | 70% | Asymptotic to 0.025%† |
| | 99% | Asymptotic to 0.011%† |
| 10%/10% | 10% | 0.14%† |
| | 50% | Asymptotic to 0.023%† |
| | 90% | Asymptotic to 0.278%† |

†High probability of resistance allele going to extinction.

Wherein population crashed means that in the experiments the population went to extinction. No individuals in the population survived after a given exposure to the toxins.

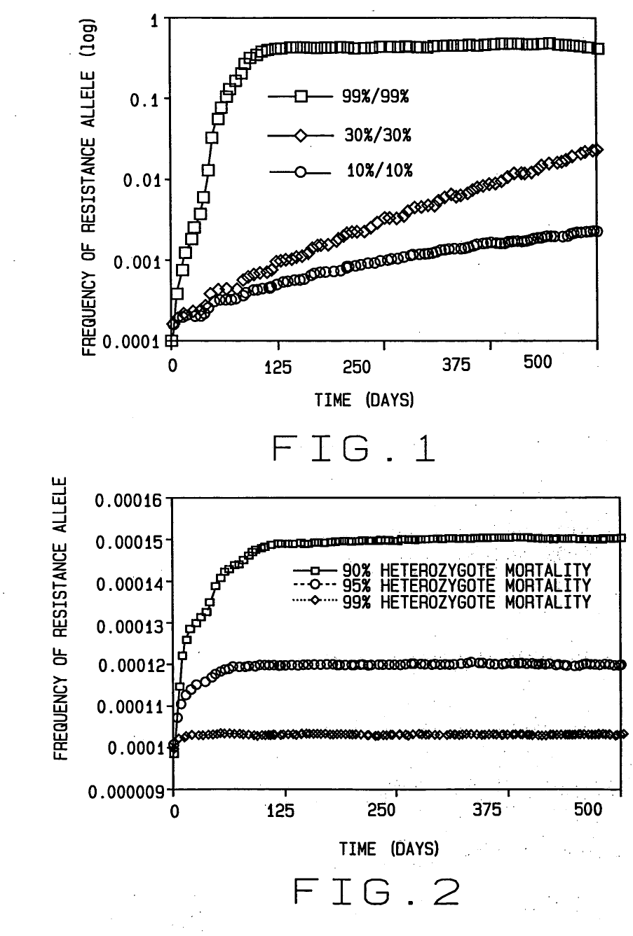

FIGS. 1 through 4 graphically illustrate the concurrent use of two toxins that are negative cross resistance factors on a target pest population, such as an insect population. If the combined toxins kill more heterozygotes than homozygotes, the allelic frequency of mutant alleles tend to go to an extreme value, i.e., either close to fixation or close to extinction depending on initial frequency. In FIG. 1, both toxins cause equal mortality in homozygous wild-type and mutant pests, but the heterozygotes are unaffected by the combination (1% heterozygous mortality). In FIG. 2, the two toxins cause equal mortality rates in the homozygous lines and the heterozygotes have higher mortality rates than the homozygotes.

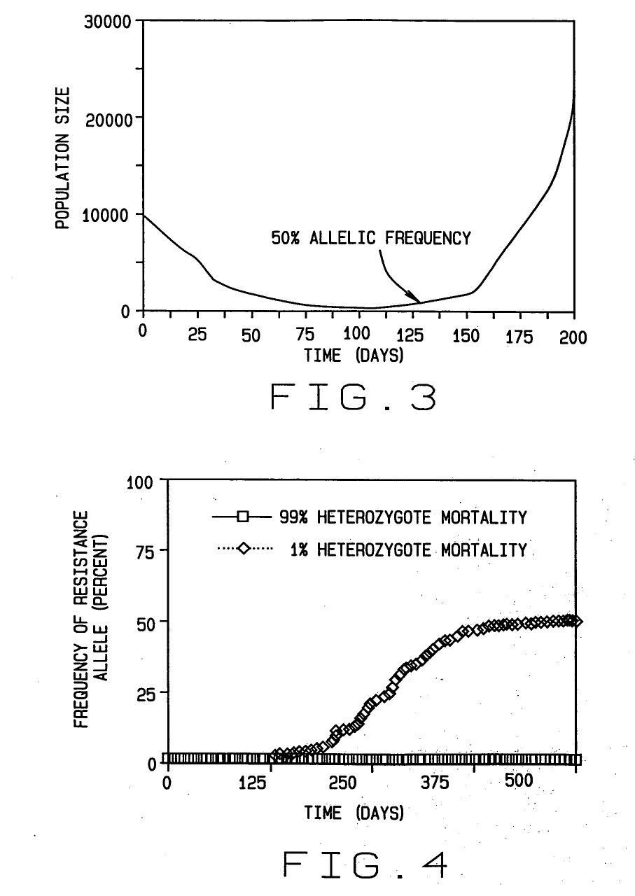

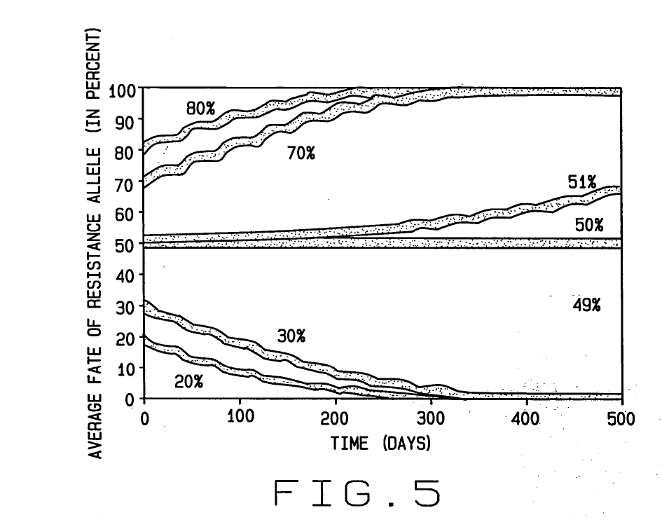

When the combined toxins kill fewer heterozygotes than homozygotes, the allelic frequency of the two alleles approaches a 50% equilibrium point. In FIG. 3, the first toxin causes a 99% mortality rate to the homozygous wild-type pests, and the second toxin causes a 70% mortality rate to the homozygous resistant pests. In addition, 70% of the heterozygous target population are killed (in addition to the background mortality rate). As illustrated in FIG. 3, when the frequency of the resistance allele becomes greater than 50%, the population begins to expand. In FIG. 4, both toxins cause a 70% mortality rate in the homozygous wild-type and mutant pest population, but the heterozygotes are either unaffected by the combination (1% heterozygote mortality) or they are much more susceptible (99% heterozygote mortality).

If the allelic frequency of the two alleles approaches a 50% equilibrium, the pesticide combination is not effective in controlling the pest population. It is important to note that high heterozygous fitness as compared to both homozygotes is rare in nature. Since heterozygous fitness in the presence of both toxins has not typically been investigated in field and laboratory examples of NCR it is difficult to assess how rare the phenomenon is. In cases where high heterozygote fitness is observed in screens to discover NCR factors, such toxins should typically be given low priority for commercial development.

High heterozygote mortality rates result in the resistance allele becoming very rare in the population or extremely common in the population, depending on the starting allelic frequency. Since the frequency of the resistant allele is an issue of probability in a field situation, in which there are many variables, it is quite possible that with a starting allelic frequency (for the resistance allele) below 50%, the resistance allele may occasionally tend to high frequency when the two toxins are used. Thus, in a field scenario, it is beneficial to initially use a single toxin to drive one allele to a more extreme value, to increase the probability of a preferred allele being the most common.

For example, assume that there is a practical reason that requires keeping the resistance allele at a low frequency. If the starting allelic frequency of resistance is greater than 10% one could use the second NCR factor (which kills the resistant pests preferentially) to reduce the frequency of the resistance allele before using the two toxins concurrently. Thus, by first applying the toxin that preferentially kills the resistant pest, one biases the probability of keeping the resistance allele at a low frequency when the two toxins are used together (See, for example, FIG. 5). Once resistance is driven to a low frequency, the single toxin can again be used. Specifically, FIG. 5 illustrates the effect of starting allelic frequency on the fate of the resistance allele in the presence of two negative cross resistance factors that kill both homozygous lines (susceptible and resistant) at a 50% level and the heterozygotes at a 70% level. The X-axis represents the time (in days) from the start of the experiment and the Y-axis represents the average fate of the alleles (replicate of 10,000 with a starting population of 10,000) from the given starting frequency shown in FIG. 5 for each of the lines.

If resistance to the first factor is recessive, the first NCR toxin is used on its own, until the resistance allele becomes common enough in the population that the first toxin is no longer effective in controlling the population. At this time, the toxin pair is then used on the pest population such that the allelic frequency of resistance moves back to a low level. Using the second NCR factors sparingly may ultimately be more economically acceptable than the continuous use of two toxins. Thus, as seen from the preceding Figures, resistant alleles can be managed in a target pest population.

### Screening for Negative Cross-Resistance Factors

The current generation of pesticides includes toxins isolated from bacterial broths, such as Spinosad, and transgenic plants containing genes that code for a pesticidal protein. As used herein, the term “transgenic” includes having a chromosome or chromosomes into which one or more heterologous genes or genes from another strain or the same species have been incorporated either naturally or artificially into an organism. Heterologous is defined as derived from a different species. There is a high probability that in some cases target-site insensitivity to these new classes of pesticides occur in the pests. Target-site insensitivity is a major mechanism of resistance to second generation pesticides. One can define second generation pesticides as those pesticides including, but not limited to, chlorinated hydrocarbons, pyrethroids, carbamate, and organophosphates. After deploying these novel toxins, it is likely that a single (or multiple) point mutation in the gene coding for the target site in the pest results in the pests developing field resistance. Additionally, metabolic resistance may occur where the pests have a greater ability to alter the toxin such that it has reduced toxic activity. Even if metabolic resistance occurs to such resistance factors, the metabolic resistance does not rule out the possibility of developing NCR compounds for control of metabolic insecticide resistance.

Present pesticide and antibiotic discovery involves the routine (and often automated) screening of tens or hundreds of thousands of candidate toxins against a repertoire of pests (and bacteria in the case of antibiotic discovery). Once a toxin has been deployed, and resistance arises, there have been few cases where researchers have made an effort to determine the existence of NCR factors.

In spite of the lack of large-scale screening for NCR toxins, there still has been discovery of such compounds. For example, it has been found that AaIT, a protein isolated from Scorpion toxin, provides NCR to pyrethroids in knockdown resistance (kdr) flies. In addition, a NCR factor to aphids has been identified that was resistant to insecticides through increased production of a carboxylesterase, E4.

Although NCR factors do occur within classes of toxins, there is no reason to believe that NCR factors will only be found in the same or substantially the same class of compounds as the first toxin. Although compounds within the same or substantially the same classes of toxins appear to be a logical starting place, exemplary screens for NCR toxins involve random screens for compounds. The random screens are, in one embodiment, coupled with a ‘clue-based’ screen.

### Method of Screening for Negative Cross-Resistance Factors

An advantage of random screening for NCR factors is that an understanding of the molecular basis of resistance is not necessary for the development of the second compound. Although knowledge about the molecular basis of resistance typically lags years behind the first appearance of resistant pests in the field, knowing the basis of resistance is helpful for ‘clue-based’ screening. However, if discovery of the molecular basis of pesticide resistance is too costly or time consuming, one may be able to use the resistant line (or lines) in a random screen for NCR factors.

Tests using resistant and susceptible lines of pests are easily integrated into current large-scale automated screening methodologies. The screens identify compounds that are toxic to the resistant line (or lines) in the bioassay and not toxic to the pest lines that are susceptible to the already commercialized toxin.

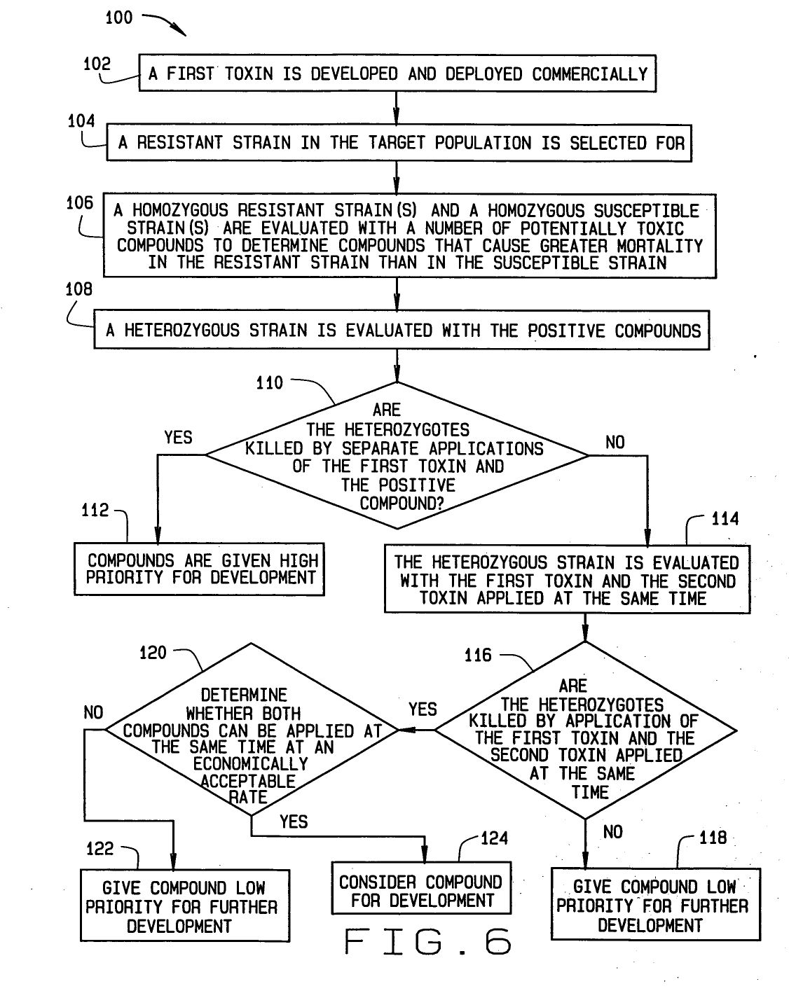

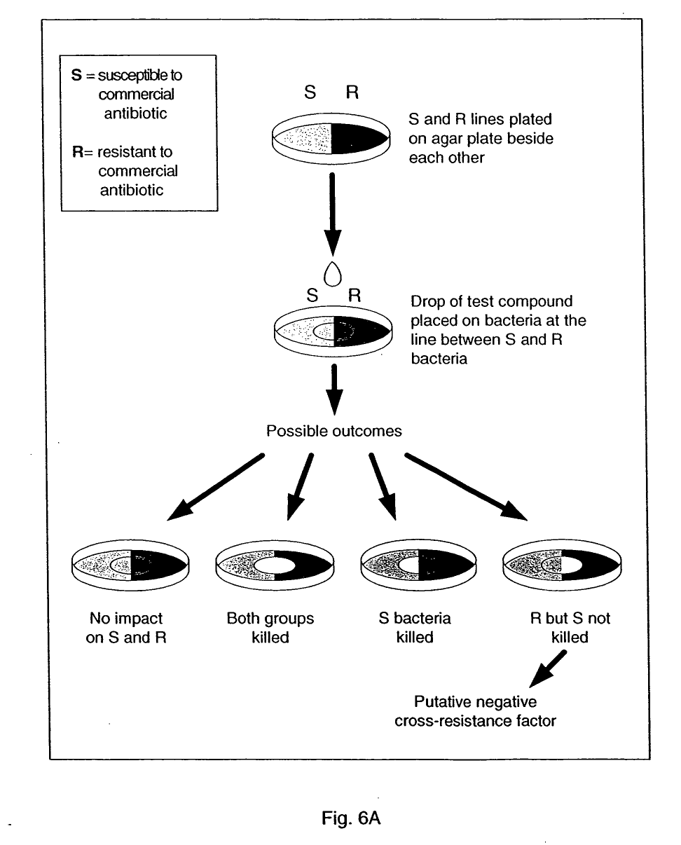

FIG. 6 illustrates a method 100 of evaluating the efficacy of compounds to be deployed commercially as NCR factors. A first toxin is developed and deployed 102 commercially. The first toxin is lethal to at least a portion of a target population, e.g., an insect population, a weed population, a bacterial population; a fungus population, a virus population, and a population of organisms living within a body of a higher order mammal. A susceptible strain (S/S) of the target population is determined that is susceptible to the first toxin. After the first toxin has been used for a period of time, a strain of the target population that is resistant to the first toxin begins to develop and grow. The resistant strain is selected 104 for use in NCR evaluation. In one embodiment, the resistant strain is selected for using a field collected strain. In an alternative embodiment, the resistant strain is selected for using an EMS-mutagenized line, a P-element mutagenized line, or any other mutagenized genomic line selected for pesticide resistance. As used herein, the term “EMS-mutagenized line” includes the use of ethyl metanesulfonate to generate mutations in the genome of an organism that will be used to discover or determine NCR in toxins.

Illustratively, an EMS-mutagenized line is prepared by feeding male Drosophila with a sugar solution containing ethyl metanesulfonate and crossing these with female Drosophila. The progeny of this of further crosses can then be bio assayed for resistance or susceptibility to a toxin. One of the disadvantages of selection through mutagenized lines is that mutants may be found that confer resistance to the toxin, but the alleles may not be commonly observed in nature.

For example, DDT and pyrethroid resistance mutations have been identified in regions of the Drosophila sodium channel, para, that have not been observed in nature. One of the resistance mutation lines studied showed no NCR to AaIT, but the naturally occurring kdr (from field lines of insects) showed 9 to 14-fold more susceptibility to this compound. Thus, the use of mutagenized lines to screen for negative cross resistance in large-scale bioassays may result in failing to identify compounds that may be effective in resistance found in the field. Alternatively, one may also identify NCR factors from mutagenized lines that may not be ultimately useful in field resistant lines of insects.

After the resistant strain has been selected for, a homozygous resistant strain (R/R) is evaluated 106 with a number of potentially toxic molecules, e.g., natural molecules, synthetic molecules, chemicals, compounds, biotechnical species, and biotechnical moieties, to determine a second toxin that is more toxic to the resistant strain (R/R) than to the susceptible strain (S/S). In one embodiment, a screen is conducted for negative cross-resistance in the R/R lines only. In another embodiment, a screen is conducted for negative cross-resistance in the heterozygous resistant (R/S) lines only. In a further embodiment, a screen is conducted for negative cross-resistance in S/S and R/S or R/R and R/S or in combination with S/S or R/S. Heterozygous resistant insects are ones that carry one copy of the resistance allele (R) and one copy of a susceptible (S) allele. Screens of S/S lines with the toxins can occur before or after the R/S or R/R screens or S/S may not occur at all. Screens for only R/R or R/S or S/S or any combination thereof can take place in separate time or space. In one embodiment, the toxic molecules include variants, mutants, metabolites, and derivatives.

In addition, a susceptible control strain (S/S) is also evaluated with the same or substantially the same compounds. In one embodiment, the strains are evaluated with between about 10 and 10⁹ compounds. In an alternative embodiment, the strains are evaluated with about 10² to about 10⁸ compounds. In a further alternative embodiment, the compounds are evaluated with about 10³ to about 10⁷ compounds. In one embodiment, compounds to be screened include chemicals from known pesticides, insect biocides, and their variants, mutants, metabolites, and derivatives. Exemplary chemicals include and are not limited to a) Bacillus thuringiensis proteins and their variants, b) clorinated hydrocarbons, c) organophosphates, d) pyrethroids, e) carbamates, f) variants of toxins from the bacteria Photorhabdus luminescens, g) insect growth regulators and their derivatives, h) alpha-amylase inhibitors, i) lectins, j) Spinosad derivatives, k) spinosyns and their derivatives, l) derivatives of insecticidal compounds from the bacteria Saccharopolyspora spinosa, m) Bacillus thuringiensis strains and their variants, n) protease inhibitors and their derivatives, o) Cysteine protease inhibitors and their derivatives, p) Bowman-Birk Inhibitors and their derivatives, q) Kunitz inhibitors and their derivatives, r) Saccharopolyspora spinosa strains and derivatives of their insecticidal and non-insecticidal toxins, and s) imidacloprid or derivatives of imidacloprid.

In a further embodiment, molecules are supplied from randomly or selectively generated chemicals, and random or selective (chemical rationale approach) screening of chemicals. The molecules to be evaluated further include molecules supplied from bio-prospecting from plant, animal, bacteria, and fungal organisms or extracts of these organisms and from prokaryotic or eukaryotic organisms. The molecules to be evaluated also include molecules supplied from the generation of antibodies showing preference for binding to proteins or protein complexes or membranes in the organism involved in negative cross resistance (binding preference for versions of the protein that are resistant to the first toxin) and generation of random peptide libraries and bio-panning using phage display. A random peptide library is made and is screened for affinity to the product of the target of interest, e.g., the gene product of the target site. The resistant allele, more specifically the protein product, is then used to identify a protein that has high affinity to the gene product to generate a NCR toxin for specifically targeting the resistant pest. The molecules to be evaluated also include molecules obtained from combinatorial shape libraries and molecules supplied using combinatorial chemistry.

The above described compounds are exemplary only and are not intended to limit the compounds to only those described above. In addition, although method 100 describes evaluations, it should be understood by one of ordinary skill in the art that screening methods can be utilized for the evaluations.

Those compounds that are more toxic to the resistant strain than to the susceptible strain are considered to be positive compounds for the initial evaluation. A heterozygous strain (R/S) of the target population is evaluated 108 with the positive compounds to test their effectiveness against the heterozygous insects. Thus, the resistant (R/R) and susceptible (S/S) pests are crossed and the progeny bio-assayed against the new toxin. It should be determined whether resistance is sex-linked where appropriate), since if the resistance is sex-linked, individuals of the proper sex that carry two alleles of the gene should be used. For example, since Drosophila is XY for males and XX for females, a bioassay in this species for sex linked resistance should initially focus on females. (XY=female and male sex chromosomes and XX=two female sex chromosomes) The heterozygotes are screened by using separate applications of the first toxin and the positive compound being tested, i.e., the second toxin, to determine if separate applications of the first toxin and the second toxin are at least as toxic to the heterozygous strain (R/S) as to the susceptible strain (S/S) of the target population.

If the heterozygotes are killed by separate applications of the first toxin and the positive compound being tested 110, the positive compound is given a high priority for development 112 and commercial exploitation. A high negative cross resistance priority is assigned to the second toxin if separate applications of the first toxin and the second toxin are at least as toxic to the heterozygous strain (R/S) as to the susceptible strain (S/S). Thus, based on the toxicity of the compound to heterozygous individuals, the practical applicability of each toxin is prioritized and the compounds capable of killing the heterozygotes receive a high priority while those compounds that only impact homozygous individuals are subjected to further testing and evaluation to determine their prioritization. The priority compounds, in one embodiment, are prioritized for advancement to additional evaluations which are utilized to make commercial development prioritization decisions. In an alternative embodiment, the high priority compounds receive a commercialization prioritization.

The heterozygous strain is evaluated 114 with the first toxin and the second toxin applied at the same time to determine 116 if application of the first toxin and the second toxin at the same time is at least as toxic to the heterozygous strain (R/S) as to the susceptible strain (S/S). If the application of the first toxin and the second toxin at the same time is not at least as toxic to the heterozygous strain as to the susceptible strain 116, the compound is given a low priority for further development.

In an alternative embodiment, the second toxins are prioritized based on their performance in the resistant strain evaluation and in the heterozygous strain evaluation. At least one of the highest prioritized second toxins is selected for advancement to additional evaluation to determine a commercialization prioritization.

If the application of the first toxin and the second toxin at the same or substantially the same time are at least as toxic to the heterozygous strain as to the susceptible strain 116, then a determination is made regarding whether both compounds can be applied 120 at the same or substantially the same time at an economically acceptable rate. If both compounds cannot be applied at the same or substantially the same time at an economically acceptable rate, the compounds should be given a low priority 122 for further development. If both compounds can be applied at the same or substantially the same time at an economically acceptable rate, the compound should be considered 124 for commercial development. For example, an economically acceptable rate is, in one embodiment, a rate someone is willing to pay for using the compounds to obtain a desired effect on the target population.

In one embodiment, a high negative cross resistance priority is assigned to the second toxin if the first toxin and the second toxin can be applied to the target population at the same or substantially the same time at an economically acceptable rate. For compounds that test positive for application at the same or substantially the same time, the second toxin, in one embodiment, is applied every time the first toxin is applied. In an alternative embodiment, the second toxin is applied intermittently with application of the first toxin, e.g., the second toxin is applied every other time the first toxin is applied. In an alternative embodiment, the second toxin is applied intermittently with application of the first toxin, e.g., the second toxin is applied less frequently than every other time the first toxin is applied. In an alternative embodiment, the second toxin is applied intermittently in the absence of the first toxin once or several times before the first toxin is again applied.

Thus once a pair of NCR factors is determined and identified, many different types of applications of the toxins to the insects can be used. For example, both toxins can be applied at the same or substantially the same time every time, one of the toxins can be applied on an intermittent basis, both toxins can be applied on an intermittent basis, and the toxins can be applied in an alternating type application. In one embodiment, the toxins are delivered to the target population utilizing at least one of sprays, pellets, powders, baited or non-baited traps, and transgenic organisms. The toxin or toxins can be delivered in organic or inorganic solvents or in an aqueous solution. Organic solvent refers to a group of volatile mixtures or compounds, which are relatively stable chemically and that exist in the liquid state at temperatures of between about 32° to about 482° F. (0° to 250° C.). Most organic solvents are classified as ketones, aliphatic hydrocarbons, cyclic hydrocarbons, aromatic hydrocarbons, aldehydes, halogenated hydrocarbons, amines, esters, alcohols, and ethers. Inorganic, in the context of chemistry, refers to substances that do not contain atoms of carbon. A solvent is, in one embodiment, a liquid substance, capable of dispersing or dissolving one or more other substances. An example of an inorganic solvent is water. An aqueous solution is any solution containing water.

For example, in the case of weeds, a first compound is applied to the field by spraying the compound on the weeds using an inorganic, an organic, or both solvents. If resistant forms exist in the particular field, a second compound is then applied to the field. In addition, transgenic antibodies or antibody conjugates with toxins attached could be used in the selection assays. Thus, in one embodiment, the above described method is used to manage a tract of land against a resistant strain of a target population. In an alternative embodiment, the above described method is used to control an insect population on a tract of land where it is desired to control the insert population. In a further alternative embodiment, the above described method is used as part of a pest management system to manage a pest population such as a strain of fungus or a strain of bacteria where it is desired to manage the pests.

Although the evaluations described above are described in the context of whole organism screens, the above described method is not limited to whole organism screening. For example, components from the insects could be used for adaptations of in vitro screens for target sites. It is known that insects develop resistance to plant inhibitors and bio-panning helps to develop new forms of inhibitors useful in control of the insects.

In an alternative embodiment, a compound is selected in vitro that works better at the resistant target site than the susceptible target site. This compound is then subjected to the screening process described above to determine if the compound is a viable NCR factor.

In a further alternative embodiment, the above described method is utilized to select for a first virus that kills the insects resistant to a second virus. The viruses are made transgenic in plants. Eventually, the insects become resistant to the first virus. The above described method is then used to evaluate and identify variants of the viruses or other related viruses.

In a further alternative embodiment, the assay system can be adapted for tests with resistant microorganisms. FIG. 6A illustrates a method of evaluating the efficacy of compounds to be deployed commercially as NCR factors in the control of microorganisms. A first toxin is developed and applied. The first toxin is lethal to at least a portion of a target population, e.g., a microorgansm. A susceptible strain (S) of the target population is determined that is susceptible to the first toxin. After the first toxin has been used for a period of time, a strain of the target population that is resistant to the first toxin begins to develop and grow. In an alternative embodiment, the resistant strain is created in the laboratory using mutagenesis techniques. The resistant strain is selected to be used in a NCR evaluation. The resistant and susceptible microorganisms can be assayed to evaluate the ability of compounds to limit the growth or increase the mortality of the resistant strain. Limit the growth is defined, but is not limited to, as a reduction of growth of the microorganism on a solid medium or in a liquid medium. An example of a solid medium is that of an agar or agarose plate. An example of a liquid media, but not limited to, is that of Luria-Bertani (LB) medium. LB medium is a serial solution containing 10 grams of tryptone, 5 grams of yeast extract, and 10 grams of NaCl per liter of solution.

In an alternative embodiment, the resistant strain or microorganism is selected for using Chemical mutagenesis (e.g., but not limited to, EMS-mutagensis), transposable elements, sit-directed mutagenesis, gain of function mutants, loss of function mutants, or any other mutagenized genomic line selected for antibiotic resistance.

Another form of negative cross-resistance is exemplified with DDT resistance in Drosophila melanogaster (Drosophila) and the compound phenyl-thio-urea (PTU). When PTU is fed to metabolically resistant Drosophila, the cytochrome P450 enzymes causing resistance to DDT bio-activate the PTU into a toxic compound. The DDT-resistant Drosophila line has higher mortality and takes longer to develop than the susceptible flies. In an alternative embodiment of the invention, potential NCR compounds are screened to determine if the toxins impact other life-history parameters, e.g., delays in developmental time, reduction of the fecundity of only the resistant insect lines. If a NCR toxin is used that delays developmental time, the resistance allele can be kept at a lower level in the population.

Exemplary insect lines or strains that can be used to screen for compounds include, but are not limited to, Drosophila lines or strains including Rst(2)DDT-Wisconsin and Rst(2)DDT-Hikone. Both lines or strains exhibit NCR between DDT and PTU. More particularly, Rst(2)DDT (also known as Dimethylnitrosamine demethylase; Dmnd) exists between cinnabar (cn) and vestigal (vg) and evidence exists to suggest that the Rst(2)DDT locus is the gene CYP6G1 (a cytochrome P450 gene) and resistance is due to over-expression in the Rst(2)DDT-Wisconsin fly line. For example, Rst(2)DDT is located at the cytological position 48A-49D (Recombination map position 64.5-66.0 on Chromosome 2). The position of CYP6G1 is 48F1(Cytological position) and it thus lies in the middle of the resistance region. See for example, FlyBase (http://cbbridges.harvard.edu:7081/).

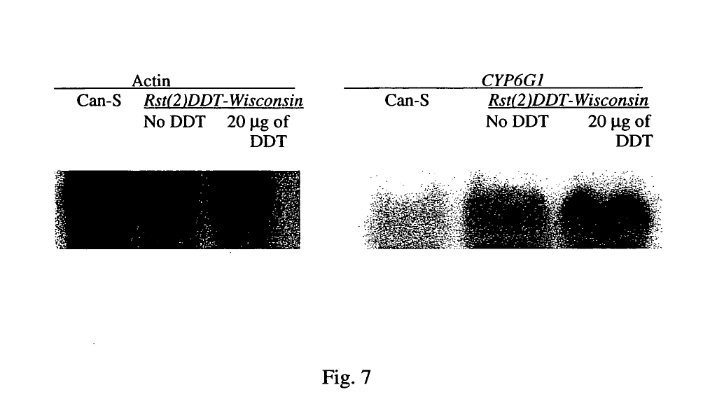

FIG. 7 is a Northern blot of a Canton-S (Can-S), DDT-resistant line with no exposure to DDT (Rst(2)DDT-Wisconsin), and a DDT-resistant fly line with exposure to 20 μg /vial of DDT for 24 hours (Rst(2)DDT-Wisconsin-20 μg of DDT). The blot was probed with actin and CYP6G1 cDNA.

Total RNA was extracted from the flies, and run on a 1.5% agarose gel for 3.5 hours. The RNA was transferred to a nylon membrane, and the membrane was probed with P32 labeled gene fragments in a hybridization solution overnight. The blots were washed in a 2×SSC (+0.1% SDS) solution three times at 15 minutes per wash and in a 2×SSC (+0.1% SDS) solution three times at 15 minutes per wash. The blots were then exposed to X-ray film for a time period that clearly showed the bands with a minimal amount of background.

Resistance has been associated with increased expression of cytochrome P450 genes, as is typical of metabolically resistant insects. In the resistant line, over-expression of CYP6G1, which had >10-fold expression as compared to the susceptible fly line (Canton-s), was observed. Evidence exists to suggest that the Rst(2)DDT-Wisconsin flies dramatically over-express resistance. Resistance may be due to at least one of over-expression in the resistance line (genotype) and over-expression and a mutation in the CYP6G1 gene in the resistance line (genotype).

In one embodiment of the invention, the above described method is utilized with an Rst(2)DDT fly line to develop chemicals that selectively target resistance. If this mechanism of resistance is common to other species, metabolically resistant Drosophila can be used to screen for NCR toxins that reduce metabolic resistance in other insect species. In an exemplary embodiment, the screening method is used to identify toxins to minimize metabolic resistance in mosquitoes that transmit malaria. Rst(2)DDT is known to confer resistance to DDT, chlordane, lindane, and imidacloprid and Rst(2)DDT can be targeted to develop toxins to control metabolic resistance to one or all of these compounds.

In alternative embodiments, the above described methods are utilized with putative target sites other than Rst(2)DDT. Exemplary putative target sites include, but are not limited to, acetylcholinesterases, voltage-gated sodium channels, GABA receptors (Rd1 or Resistance to Dieldrin (1,2,3,4,10,100Hexachloro-6,7-epoxy-1,4,4a,5,6,7,8,8a-octahydro,endo,exo-1,4:5,8-dimethanonaphthalene>in Drosophila is an example), esterases, cytochrome P450s, neurotransmitter uptake channels, cation and ion channel aromatic biogenic amine receptors, and Glutathione-S-Transferases. Another putative target site is CY6G1 in Drosophila also currently referred to as (a) CG8453, (b) AF083946, C6G1_DROME, AC Q9V674 and O76800 in gene nomenclature.

Several resistant lines, with potentially different forms of resistance can be incorporated into the bio-assays. Thus screens can be conducted for a series of toxins before resistance becomes problematic in the field, and when resistance occurs in the field, at low frequency, this repertoire of toxins can be tested against the emerging resistant lines. Other management strategies can be incorporated into field practices to slow the rate of entry of these resistance alleles into the population while there is time to develop the NCR factor to a commercial level.

In a further embodiment, a screening methodology is used to screen bacterial strains to determine NCR toxins capable of causing greater retardation of growth, or even mortality, of the resistant bacteria as compared with susceptible bacteria. An illustrative strain in Drosophila melanogaster would be the Rst(2)DDT-Wisconsin line of flies that shows increased resistance to DDT but negative cross resistance to phenylthiourea. In the exemplary embodiment, both resistant and susceptible strains are screened for NCR at the same or substantially the same time, as described above.

In addition to effecting solely the bacteria, this bacterial NCR strategy can be utilized against a higher order pest, such as an insect or a mammal since some strains of bacteria may survive only within a host organism, i.e., the bacteria live symbiotically with their hosts. Without these bacterial organisms, the host may not be able to survive. For example, bacteria live in the guts of termites. The bacteria break down cellulose consumed by the termites and convert the cellulose into energy for the termites. If the bacteria are killed, such as by a compound, the termites will be detrimentally effected. A detrimental effect includes, but it not limited to, high mortality rates of individuals in a colony or the whole colony or termites. The above described screening methodology can be utilized to screen for NCR toxins that are effective, not only against the bacteria but also against the host in which the bacteria live. Thus an NCR strategy can be used to control a pest population without directly targeting the pest, but instead by targeting an organism living within the pest.

In a still further embodiment, a screening methodology is used to screen for herbicide resistance to determine NCR toxins capable of causing greater retardation of growth or mortality of resistant plants as compared with susceptible plants. In the exemplary embodiment, both resistant and susceptible strains are screened for NCR at the same or substantially the same time, as described above. In an alternative embodiment, the screen is adapted to cover not just diploids, but also, other ploidy levels.

In another embodiment, a screening methodology is used to screen for nematodes resistant to toxins used to control them. The screens are used to determine NCR toxins capable of causing greater retardation of growth or mortality of resistant nematodes as compared with susceptible nematodes.

In a further embodiment, a screening methodology is used to screen for NCR toxins against recently identified Bt (Bt=Bacilluo theringiensis toxin) resistance alleles. It is known that Bt resistance can occur due to a truncation of a gut cadherin gene or when the cadherin gene is not glycosylated. The resistant lines are BtR-4 in H. Vireizsceiz and Bre-5 in Caenorliabditis elegans (a nematode). These lines can be used in a methodology similar to that described above to discover NCR toxins for use in Bt resistance to insects and to nematodes.

In yet another embodiment, a screening methodology is used to screen for fungicide resistance to determine NCR toxins capable of causing greater retardation of growth or mortality of resistant fungi as compared with susceptible fungi. In the exemplary embodiment, both resistant and susceptible strains are screened for NCR at the same or substantially the same time, as described above.

In one embodiment, large-scale screens are utilized to determine NCR toxins that can be used against fungicide resistance. For such a screen, there will be one of three outcomes: (1) positive cross-resistance, (2) neutral (no cross-resistance), and (3) negative cross-resistance. Table 3 illustrates the EC₅₀ for each toxin as given on two lines of Mycosphaeralla graminicola. The EC₅₀ is the effective concentration that will kill fifty percent of the organisms in a test population. One line is Triadimenol susceptible and the other line is resistant to this compound. An estimate is made of the nature of resistance of these two lines to nine other toxins. The resistance is defined as (i) positive cross-resistance, (ii) negative cross-resistance or (iii) no cross-resistance (neutral) as compared to the resistance to Triadimenol (1RS,2RS;1RS,2RS)-1-(4chlorophenoxy)-3,3-dimethyl-1-(1H-1,2,4-trizol-1-yl) butan-1-o. Such a comparison is useful in a large-scale screen for NCR toxins for fungicides.

TABLE 3

| | Mycosphaeralla graminicola | | Resis- | |
|---|---|---|---|---|
| | Wild type | Tri R1 resistant | tance | Resistance |
| Fungicides | Strain EC₅₀ | Strain EC₅₀ | Ratio | Type* |
| Triadimenol | 0.30-0.40 | 10-12 | 31 | Standard |
| | | | | for Com- |
| | | | | parison |
| Flusilazole | 0.005-0.008 | 0.020-0.030 | 4.2 | Positive |
| Tebuconazole | 0.009-0.15 | 0.035-0.0045 | 3.3 | Positive |
| Fluquinconazole | 0.0035-0.0045 | 0.001-0.002 | 0.4 | Negative |
| Prochloraz | 0.001-0.002 | 0.004-0.006 | 3.3 | Positive |
| Triflumizole | 0.002-0.004 | 0.0008-0.0012 | 0.3 | Negative |
| Imazalil | 0.004-0.006 | 0.020-0.030 | 5.0 | Positive |
| Ferarimol | 0.035-0.045 | 0.009-0.012 | 0.3 | Negative |
| Nuarimol | 0.050-0.060 | 0.020-0.025 | 0.4 | Negative |
| Fenpropimorph | 0.050-0.070 | 0.030-0.070 | 0.9 | Neutral |

*Considered as potentially positive, neutral, or negative cross-resistance. Not tested statistically.

Three of the compounds listed in Table 3 are indicated as potential NCR toxins (Triflumizole, Ferarimol, and Nuarimol). In one embodiment, screening resources are used efficiently, i.e. the potential for identifying false-positives is minimized, by focusing on investigating potential NCR toxins through a test with a correction factor, such as a Bonferroni correction. Alternatively, other correction factors can be used including, but not limited to, Duncan's method. The three potential NCR positives (Triflumizole, Ferarimol, and Nuarimol) and a standard reference compound (Triadimenol), are investigated and if the confidence intervals between the two fungi lines for a given toxin do not overlap, differential toxicity can be statistically inferred. Where differential toxicity occurs between the two fungal lines on a given toxin, a difference in toxicity between the two lines is determined. If the fungal line that was resistant to the first toxin (e.g. Triadimenol) is now more susceptible to the new toxin (e.g. Triflumizole, Ferarimol, or Nuarimol) potential NCR toxin pairs are identified.

Bonferroni Correction and Duncan's Method are both mathematical means of controlling the experimentwise error when performing multiple comparisons. Bonferroni Correction is highly conservative. It has a lower false positive rate than other methods; however, it has lower power to detect significant difference between two samples. References: Analysis of Messy Data, Volume 1: Designed Experiments Milliken and Johnson. Chapman & Hall, NY, ISBN 0-534-02713-X (v. 1) 1992; Linear Regression Analysis; G.A.F. Seber, John Wiley & Sons, Inc., ISBN 0-471-01967-4; 1977.

Differences between toxins, in terms of NCR activity, may be indicated where such differences may not truly exist (type I error in statistical hypothesis testing). In the present example, the confidence interval is extended using, for example, Bonferroni correction, which is needed to control for false positives in detection of NCR toxins in large-screen tests. A NCR factor is declared if the second toxin requires a lower dosage to kill the resistant line as compared to the wild type fungal line or lines. The consequences of a failure to use Bonferroni correction normally results in higher numbers of false positives in a large-scale screen. Toxins that are selected on the basis of no correction may still be considered as putative NCR toxins, but deserving less consideration than those identified using the correction method. A Bonferroni correction is thus performed on the three putative NCR toxins (and the reference toxin) to determine if one of the toxins is a NCR factor. This corrective measure provides a small-scale screen that shows proof of concept of a large-scale screen for NCR toxins for fungicide resistance.

Table 4 illustrates the Bonferroni corrected data (from Table 3 above) of the reference compound (Triadimenol) and the three putative NCR toxins for fungicide resistance. The correction was made based on four compounds tested, each with minimally five doses to calculate the effective concentration fifty (EC₅₀).

TABLE 4

| | Mycosphaeralla graminicola | | Possible |
|---|---|---|---|
| | Wild type | Tri R1 resistant | Resistance |
| Fungicides | Strain | Strain | Type* |
| Triadimenol | 0.25-0.48 | 8.9-13.5 | Standard for |
| | | | Comparison |
| Triflumizole | 0.0013-0.006 | 0.006-0.0015 | Neutral (n.s.)† |
| Ferarimol | 0.030-0.053 | 0.008-0.014 | Negative |
| (significant) | | | |
| Nuarimol | 0.045-0.067 | 0.017-0.029 | Negative |
| (significant) | | | |

*Compounds are declared to have no NCR activity (neutral) or to have NCR activity (negative).
†Neutral means that it cannot be inferred whether the cross-resistance is positive, negative or non-existent.

From Table 4 above it can be inferred that Ferarimol and Nuarimol are NCR to Triadimenol in a screen of these compounds. Such an approach is adapted to large-scale screens for NCR toxins for fungicides.

Active Refuge

As used herein, the term “refuge” includes an agricultural land and crop management practice of setting aside a percent of the crop in non-transgenics planting, in order to maintain susceptible alleles in the insect population.

As used herein, the term “passive refuge” includes an agricultural practice of setting aside a percent of a crop in non-transgenics, in order to maintain susceptible alleles in the insect population. No preferential selection against the resistance alleles occurs in a “passive refuge”.

As used herein, the term “active refuge” includes an agricultural practice of setting aside a percent of a crop in which transgenics containing an NCR toxin are used or where a non-transgenic area is treated with an NCR toxin, in order to maintain susceptible alleles in the insect population and select out the resistance alleles.

In another aspect of the invention an active refuge strategy is utilized to slow resistance in insect populations to toxins, such as insecticidial transgenic toxins. In typical passive refuge models, the refuge strategy involves, respectively, setting aside about 20% (with spraying the refuge with insecticides) or about 4% (no spraying) of the crop in non-transgenics, in order to maintain susceptible alleles in the insect population. The susceptible insects then mate with the heterozygous and homozygous resistant insects, resulting in heterozygous progeny that are killed if they consume transgenic plants containing such toxins as Bacillus thuringiensis. This refuge strategy provides an effective, but passive, means of delaying the onset of resistance in the insect population. FIG. 8 illustrates a passive refuge model in which the refuge does not include any toxin.

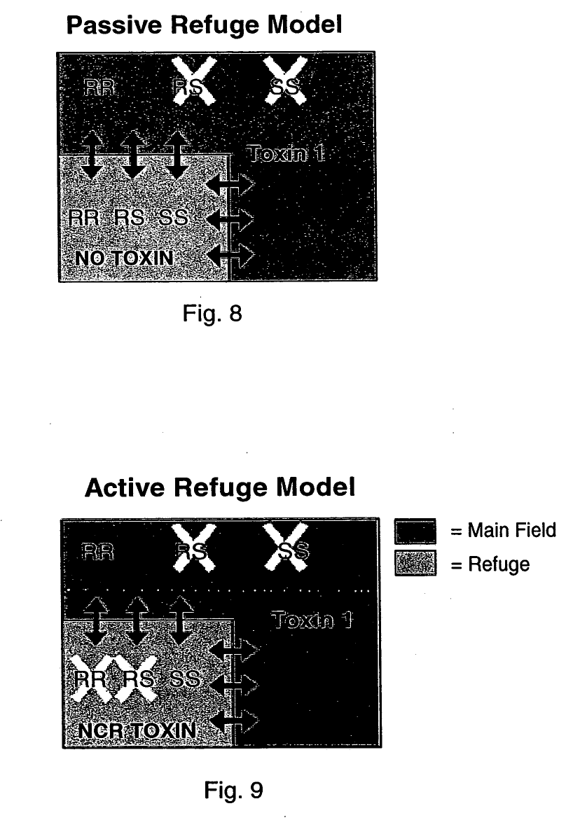

FIG. 9 illustrates an active-refuge model that employs the use of negative cross-resistance (NCR) toxins in the refuge. Instead of the refuge simply being a source of susceptible insects for mating partners with resistant insects that emerge from the transgenic field, the active-refuge uses NCR toxins to remove resistance alleles from the insect population that occur in the refuge. To date, small-scale studies have not identified Bt NCR toxins that would be useful in such a strategy. Ultimately it is believed that the discovery of NCR toxins for insecticidial toxins will require large-scale screens similar to what is currently used to discover new classes of toxins.

In another aspect of the invention, a deterministic model is used to test the hypothesis of using negative cross-resistance toxins in a refuge to slow the rate at which a major resistance allele enters the insect population. A methodology similar to that described above is utilized except that this methodology also considers selection pressure on the resistant allele in the refuge, replacing the standard HWE (Hardy-Weinberg Equilibrium) in the refuge population. The Hardy-Weinberg Equilibrium is defined as follows. The allele and genotype frequencies in a population of a particular organism remain constant from generation to generation as long as none of the following forces of evolution are occurring in the population. These forces of evolution include: genetic drift (random changes in the gene pool), gene flow (migration in and out of the population), mutation (alters alleles frequencies), non-random mating (this reduces the frequency of heterozygous individuals), and natural selection (differential reproduction or mortality will increase some alleles and decrease other alleles).

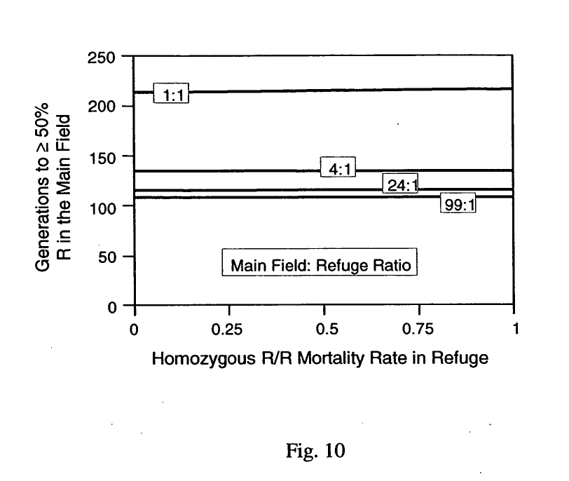

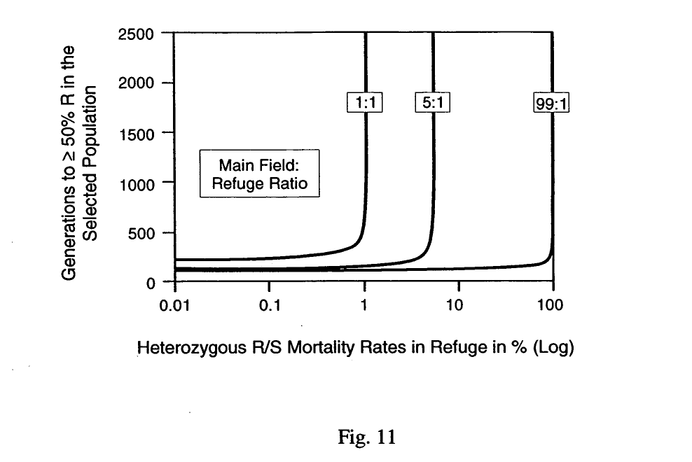

FIG. 10 illustrates the results that, if the NCR toxin is only effective in killing the R/R insects, but not the R/S insects, there is no delay in resistance as compared to the passive refuge strategy. However, as illustrated in FIG. 11, if the NCR toxin is effective in killing R/S and R/R insects, then the active-refuge can dramatically delay the development of a major resistance allele in the insect population. In addition, if the NCR toxin is marginally effective in killing insects heterozygous for minor resistance alleles (e.g. 10% R/S mortality rates) then the entry of the resistance alleles into the population is also slowed. As the NCR toxin is more effective in killing R/S insects, smaller refuges can be used to delay the development of the major resistance allele. Negative cross-resistance toxins effective against R/S or R/R insects will greatly delay the development of resistance (FIGS. 10 and 11) in an active refuge model. Additionally, since the intensity of selection directly effects the effectiveness of refuges to minimize resistance, highly selective NCR toxins should be selected. Further, low allelic exchange rates between the selected field and refuge populations may reduce the effectiveness of active-refuges, especially when the refuges are small (See Table 5). But, if the NCR toxin is capable of killing 99% of both the R/R and R/S insects, even a 1% refuge can dramatically delay resistance.

Table 5 illustrates the effects of (1) refuge size and (2) exchange rate between the main field and refuge on the number of generations to greater than or equal to 50% of R in the main field. Comparisons are made between an active refuge (R/S and R/R mortality rates of 99%) and a passive refuge (no mortality in R/S and R/R). Selection in the main field was 99% for both S/S and R/S.

Selection is defined as a force in evolution that results in differential reproduction or mortality within a population resulting in an increase in some alleles and a decrease in other alleles.

TABLE 5

| | | | Generation to |
|---|---|---|---|
| | Main Field: | Exchange | ≧50% of R in |
| Refuge Type | Refuge Ratio | Rate | Main Field |
| ACTIVE | 1:1 | 0.95 | >>100,000* |
| | | 0.80 | >>100,000* |
| PASSIVE | | 0.95 | 20 |
| | | 0.80 | 26 |
| ACTIVE | 4:1 | 0.95 | >>100,000* |
| | | 0.80 | >>100,000* |
| PASSIVE | | 0.95 | 13 |
| | | 0.80 | 20 |
| ACTIVE | 99:1 | 0.95 | 5 |
| | | 0.80 | >>100,000* |
| PASSITVE | | 0.95 | 4 |
| | | 0.80 | 7 |

*The resistance allele remained below 1% at generation 100,000.

Examples exist where NCR toxins have very little impact on the mortality rates of organisms homozygous for the susceptible (wild-type) allele (S/S) as assumed in the above described modeling experiments. In addition, examples also exist where the toxin causes low-levels or even high levels of toxicity in the susceptible population. In the latter case, an active-refuge may be only slightly better at delaying resistance than the passive refuge approach, but the NCR toxin may at least partially or completely replace the use of second generation insecticides employed in the 20% refuge strategy. Thus, such toxins would be useful in minimizing both (i) the insect population and (ii) the use of second-generation pesticides in the refuge.

The above data in FIGS. 10 and 11 indicates that the discovery and deployment of even a moderately toxic negative cross-resistance compound, in conjunction with the refuge strategy, has the potential to dramatically slow the rate at which a major resistance allele to a transgenic insecticidial toxin will enter the insect population. One consideration for these toxins is how effective the NCR toxins are at killing insects heterozygous for a major resistance allele to the first toxin. Obviously no management strategy will indefinitely postpone the onset of resistance, since insects are able to develop resistance to just about any toxin or toxin-combination used against them. In one embodiment, the active-refuge strategy dramatically increases the commercial life of transgenic toxins, where resistance in the insect population is primarily due to a major resistance allele.

In one embodiment, the refuge size is decreased with respect to currently utilized refuges due to the increased effectiveness of the refuges. In an alternative embodiment, the active-refuge model is used in conjunction with a refuge having a size similar to the size of today's refuges. Ultimately, use of an active-refuge will require (i) the discovery and commercial development of NCR toxins for use against the major resistance allele(s) of Bt (Bt=Bacillus thuringiensis toxin) and other transgenic insecticidial toxins and (ii) the development of computer simulations that take into consideration the specific life-history parameters of the pest species and the crops they attack to determine the appropriateness of the active-refuge hypothesis for these systems.

Screening for a Third Allele

In another aspect of the invention, a screen is performed to determine whether, if pairs of toxins are deployed, a third allele will arise that confers resistance to both compounds (toxins). The third allele screen is performed prior to commercial development of the second NCR toxin. In one embodiment, a screen is utilized in which toxin-pairs are applied to heterozygotes that have been EMS-mutagenized to determine alleles capable of surviving both toxins at once. If no dually resistant mutant is observed, then these toxin-pairs should be given high priority for further development. If dually resistant mutants are observed, but have high fitness costs, then these toxin-pairs should be pursued commercially.

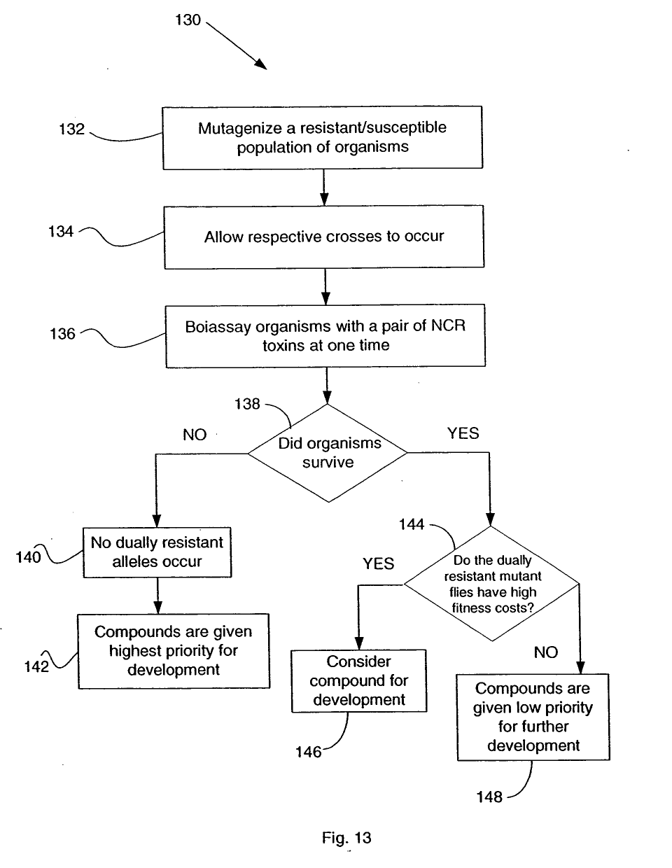

FIG. 13 illustrates a method 130 of screening for a dually resistant allele. A resistant/susceptible population of organisms, such as a population of flies, is mutagenized. The organisms are mutagenized 132 via at least one of, but is not limited to, EMS, X-rays, Gamma-rays, radioactive materials, P-elements, mobile genetic elements, or jumping genes. X-ray is a type of radiation or energy in movement that can go through solid substances and can result in the altering of heritable genetic code. Gamma-rays are defined as highly energized, deeply penetrating photons that radiate from a nucleus during the process of fission and often accompanies radioactive decay. Radioactivity is a property that is possessed by some elements (for example, but not limited to, uranium) or isotopes (for example, but not limited to, carbon 14) where they spontaneously emit energetic particles (for example, but not limited to, electrons or alpha particles) by the process where there is a disintegration of their atomic nuclei. Rays are emitted that can impact biological systems and more specifically alter the heritable genetic code of an organism. Mobile genetic elements are genetic material that can move their position on a chromosome in an organism. This movement of the mobile element in the genome can result in disruption, over expression or under expression of other unrelated gene or gene products. Genetic mobile elements can also be introduced into Drosophila melanogaster eggs by injection. Such genome altering methods in turn have the potential to change the phenotype of the organism. The phenotype is defined as an observed characteristic of an individual. A phenotype can be a result of interactions of the genotype with the environment. For example, a bioassay is performed to determine if a mutagen has given rise to a genotype that produces a phenotypic effect resulting in greater resistance to a toxin. Such examples of mobile genetic elements include, but are not limited to, P-elements in Drosophila melanogaster. A P-element in Drosophila melanogaster is a type of genetic material capable of making copies of its specific genetic material and having these copies inserted in another region of the genome. The P-element has the capacity to excise itself from one location and move to another location in the genome. Such mobile genetic elements can also be referred to as jumping genes.

Respective crosses among the mutagenized organisms are allowed 134 to occur. The resulting organisms are bioassayed 136 simultaneously with a pair of NCR toxins. It is then determined whether any organisms survive 138 the bioassay. If no organisms survive 140 the bioassay after extensive screening, there is an inference that the no dually resistant alleles are effective against the tested compounds. These compounds are given 142 a high priority for development. If organisms survive the bioassay, the surviving organisms are evaluated 144 to determine their fitness cost. If there is a high fitness cost for the surviving organisms, the compounds should be given a priority 146 for development. If there is not a high fitness cost for the surviving organisms, these compounds should be given low priority 148 for further development.

Since alternate deployment of NCR toxins has limitations, it is sometimes beneficial to utilize concurrent deployment of two NCR-toxins. If a single toxin is deployed, the toxin will kill all (or almost all) of the pests carrying susceptible alleles and the pests homozygous for the resistant alleles will survive. With concurrent two-toxin NCR deployment the same or substantially the same susceptible alleles will be targeted in addition to the major resistance allele(s). Only dually-resistant alleles (alleles that cause resistance to both toxins) will survive the two-toxin treatment. It is also likely that a dually resistant gene will be recessive and more rare than the major resistance alleles for the first toxin. High levels of resistance to toxins are often associated with mutations that impact binding of the toxin to the target protein. This resistance is often recessive and associated with a loss or change of function.

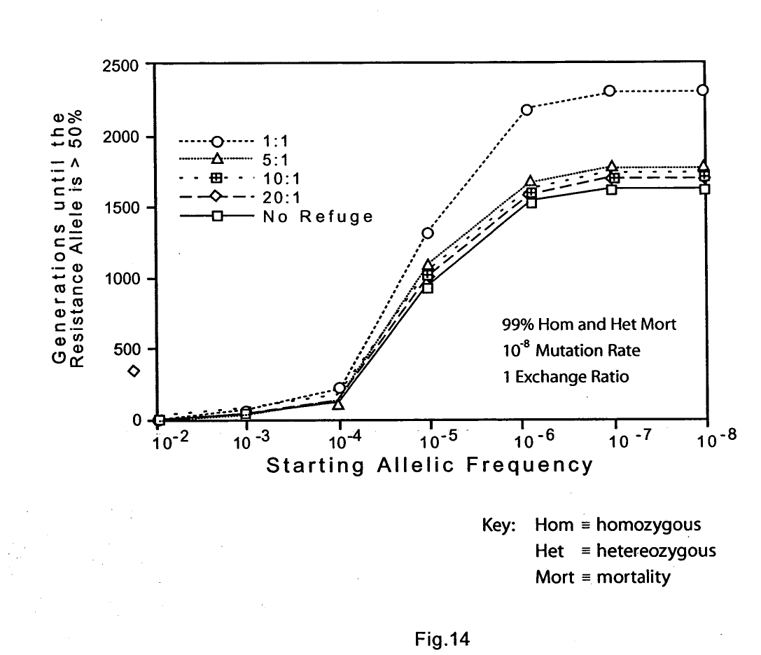

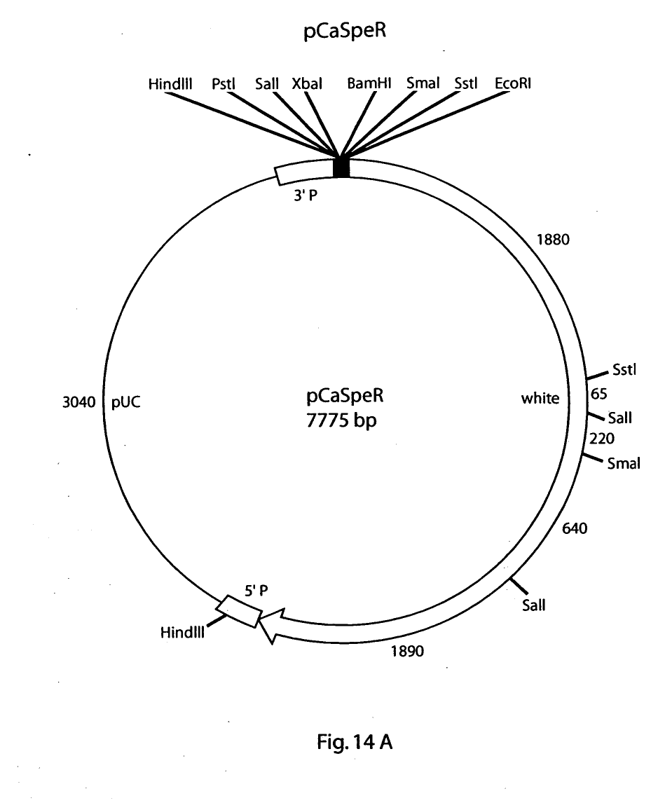

FIG. 14 illustrates the effect of the (i) starting allelic frequency (mutation rate and numbers of possible alleles that can confer resistance to the toxins) and (ii) the refuge size on the rate at which resistance enters the population in the presence of a pair of two NCR toxins. The alleles that enter the population confer resistance to both toxins. The mortality rate for the homozygous susceptible and heterozygous insects is 99%, the mutation rate was 10⁸ and the exchange rate was 1.

If a single toxin is deployed, alleles resistant to that toxin will exist in the population at a given frequency. If two toxins are deployed concurrently, i.e., a second NCR toxin is also deployed which targets the most common resistance allele, a third allele that confers resistance to both toxins should be very rare. For example, in the case of resistance to dieldrin the same or substantially the same allele occurs across a diverse group of insect taxa. This allele is a single mutation at 302 in the alpha sub-unit of the GABA receptor (19) known as resistance to Dieldrin or Rdl. In the absence of selection (prior to the use of second-generation insecticides) it is likely that this allele was rare. For example, assuming 104 alleles existed prior to selection by dieldrin, the estimated current frequency in the Drosophila population has increased about 100 fold. To date only this allele has been observed as associated with resistance to dieldrin across a great diversity of species. If a toxin were discovered and deployed that selectively killed Rdl insects, the frequency of alleles that can deal with both dieldrin and the second toxin (if such alleles even exist) would be very rare.

For example, assume that an allele which is dually-resistant to both Rdl and the hypothetical NCR toxin is present at a frequency of 10⁵ in the population. This frequency is arrived at conservatively since it can be estimated that the allele is 1/10 as common as the Rdl allele prior to selection. In reality such an allele is likely to be much less frequent. This conservative assumption provides an estimate regarding how long it will take for recessive resistance to come to high frequency in the population in the presence of a two-toxin system as compared to a one-toxin system with a refuge. If the starting allelic frequency of the dually resistant allele is 10⁵ it will take 803 generations for resistance to come to 50% frequency when the two toxins are used and no refuge is employed. Use of a single toxin with a 50% refuge results in 50% allelic frequency in 215 generations with a starting allelic frequency of resistance at 10⁴. Thus, having a negative cross-resistance toxin that selectively kills homozygotes for the most common resistance allele has the potential to effectively stave off resistance.

In one embodiment, it is beneficial to utilize refuges in combination with an NCR strategy to prevent the appearance of additional dually-resistant alleles in the insect population at appreciable levels. If the dually-resistant alleles are rare, refuges slow an increase of the dually-resistant alleles in the pest population. A determination of an appropriate size of the refuge should be made for each dual NCR-toxin system. It may be possible to use smaller refuges, than the ones currently employed, when two NCR toxins are used at once. In a further embodiment, an NCR strategy is used as a substitute to using a refuge strategy. The NCR toxins can be used as a land management practice. A land management practice is a set of tools and approaches used to grow crops such as agricultural crops.

Additionally, in the discovery of a third allele, mutatgensis of the pest population increases the probability of all or most of the resistance alleles coming into the pest population. If there are 10 possible mutations that can confer resistance in the population for a first toxin-pair (Toxin 1 plus NCR toxin 2, termed “toxins 1+2”) and one possible mutation that can confer resistance in the population to the second pair of NCR toxins (Toxin 1 plus NCR toxin 3, termed “toxins 1+3”>then it is more likely to observe the presence of dually resistant alleles for toxins 1+2 than toxins 1+3 (FIG. 13; e.g. 10⁴ alleles as compared to 10⁵ alleles). For alleles that are at a frequency of 10⁵, they will be observed much later than those alleles at a frequency of 10⁴, or 10 times the amount of putative resistant alleles can exist for this other toxin pair. A computer model has been simulated to show, at a specified population level, how rapidly resistance is observed to rise in frequency in the population. In a population of flies used in a screen, for example, resistance in a single or several generations is selected for, but FIG. 14 exemplifies that with a higher mutation rate or greater frequency of the resistance allele in the population, dually resistant alleles are observed sooner if there are more alleles that can confer resistance (e.g. 10⁴ alleles as compared to 10⁵ alleles). Mutagenesis will allow for a greater increase in frequency of point mutations and toxin pairs where more alleles confer resistance. This data suggests that a screen using NCR toxin pairs would elucidate the NCR pairs that are more likely to have resistance develop against them. Negative cross-resistance toxin pairs where few or no resistance alleles are observed should be given highest priority for development to a commercial level.

Methodology Used to Generate Computer Data For FIGS. 1, 2, 3, 4, 5, 10,

and 14

> p := (s, h, pt) -> ((1-s)*pt + (1-h*s)*(1-pt))*pt/( pt*((1-s)*pt + (1-h*s)*(1-

pt)) + (1-pt)*((1-h*s)*pt + (1-pt)) );

> p := proc (s, h, pt) options operator, arrow; ((1-s)*pt+(1-h*s)*(1-

pt))*pt/(pt*((1-s)*pt+(1-h*s)*(1-pt))+(1-pt)*((1-h*s)*pt+1-pt)) end;

> pg := (g)-> (g[1]+0.5*g[2])/sum(g[i], i=1..3);
> pg := proc (g) options operator, arrow; (g[1]+.5*g[2])/sum(g[i],i = 1 .. 3)

end;
selection := (s,h,pt) -> [(1-s)*pt*pt, 2*(1-h*s)*pt*(1-pt), (1-
pt)*(1-pt)];
selection := proc (s, h, pt) options operator, arrow; [(1-s)*pt\hat{ }2, 2*(1-
h*s)*pt*(1-pt), (1-pt)\hat{ }2] end;

> seln := proc(s,h,pt) local g; g := selection(s,h,pt); g/sum(g[i],i=1..3); end;
> mutation:= (p,m) -> p*(1-m) + m*(1-p);
> mutation := proc (p, m) options operator, arrow; p*(1-m)+m*(1-p) end;
> migration :=proc(gPopn, gRefuge, relativeSize, refugeMigration) local migr,

i, h, hPopn, hRefuge; hPopn:=[0,0,0]; hRefuge:=[0,0,0]; for i from 1 to
3 do migr := refugeMigration*(gRefuge[i] − gPopn[i]); hPopn[i] :=
gPopn[i] + migr/relativeSize; hRefuge[i] := gRefuge[i] − migr; od;
[hPopn, hRefuge]; end;

> rungen := proc(popnF, refugeF, nGen, s, h, m, relativeSize, pcntMig) local
i,

refuge, popn, popnFreq, refugeFreq, G; popnFreq := popnF;
refugeFreq
:= refugeF; for i from 1 to nGen do popn := seln(s,h, popnFreq);
refuge:=seln(0,0,refugeFreq); G := migration(popn, refuge,
relativeSize,
pcntMig); popn := G[1]; refuge := G[2]; popnFreq :=
mutation(pg(popn),m); refugeFreq := mutation(pg(refuge), m); od;
[popnFreq, refugeFreq]; end;

# How to run: # result := rungen(pPopn, pRefuge, numberOfGenerations,
# selectionPressure_s, h, mutRate, sizeRatio, #Xchange);
# result[1] gives allele freq of wild-type (targeted) allele in population.

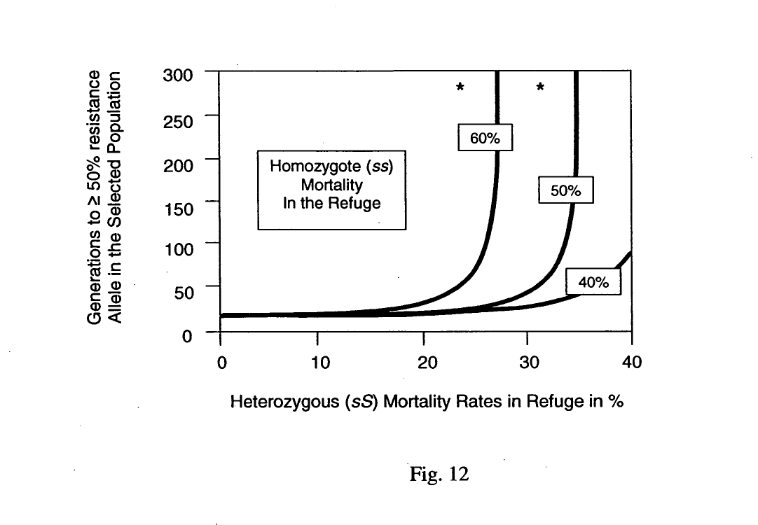

Method Used to Generate Computer Data For FIGS. 11, 12, and Table 5.

# Overall Model
Refuge_model (N, μ, p₀, L_{T}, h_{T}, K_{T}, L_{R}, h_{R}, K_{R}, z, r, F, a, b, G);
#
# Inputs

| | |
|---|---|
| # N | number of years to observe |
| # μ | winter survival rate |
| # p₀ | Initial frequency of s allele in both transgenic field and refuge |
| # L_{T}, L_{R} | fitness of ss homozygote in the transgenic field and refuge respectively |
| # h_{T},h_{R} | fitness of heterozygote in the transgenic field is h_{T}L_{T} + (1-h_{T})K_{T} and in the |
| # | refuge is h_{R}L_{R} + (1-h_{R})K_{R} respectively. |
| # K_{T}, K_{R} | fitness of SS homozygote in the transgenic field and refuge respectively |
| # z | relative preference of an insect for the toxic crop |
| # r | proportion of the population that migrate |
| # F | female insect fecundity |
| # a,b | density dependence parameters |
| # G | ratio of the size of the refuge to that of the transgenic field |
| # | |
| # Ouputs | |
| # X, Y | population densities in transgenic field and refuge respectively |
| # p,w | Frequency of s allele in transgenic field and refuge respectively |

p=p₀;
# In order to achieve equilibrium (stabilize population):
For 20 years (without selection) do;

[X,Y,p,w] = NCR_model(μ,X,Y,p,p,1,h_{T},1,1,h_{R},1,z,r,F,a,b_{,},G);

# Analysis of effect of selection over N years:
For N years do;

[X,Y,p,w] = NCR_model(μ,X,Y,p,w,L_{T},h_{T},K_{T},L_{R},h_{R},K_{R},z,r,F,a,b,G);

end;
# Sub-Routines used in Modeling
NCR_model (μ,X,Y,p,w,L_{T},h_{T},K_{T},L_{R},h_{R},K_{R},z,r,F,a,b,G);
# For a bivoltine insect, we have two life cycle events each year, followed by a
decrease # in numbers during the winter season.
# X and Y are the insect population densities in transgenic field and refuge, and p and w
# are the s allelic frequencies in these regions at year start. X_{F}, Y_{F}, P₂ and w₂
# are these quantities at year end.

| | |
|---|---|
| [X₁,Y₁,p₁,w₁] | = NCR_LifeCycle(X,Y,p,w,L_{T},h_{T},K_{T},L_{R},h_{R},K_{R},z,r,F,a,b,G); |
| [X₂,Y₂,p₂,w₂] | = NCR_LifeCycle(X₁,Y₁,p₁,w₁,L_{T},h_{T},K_{T},L_{R},h_{R},K_{R},z,r,F,a,b,G); |
| [X_{F},Y_{F}] | = NCR_WinterSurvival(X₂,Y₂,μ); |

return [X_{F}, Y_{F}, p₂, w₂];
end;
NCR_LifeCycle(X,Y,p,w,L_{T},h_{T},K_{T},L_{R},h_{R},K_{R},z,r,F,a,b,G);
# A life cycle consists of four phases: inter-field (dispersal), birth, selection and density
# dependent population correction. Two of these events change the allelic frequency
# (migration and selection). All can change the population size.
# X and Y are the insect population densities in transgenic field and refuge, and p
and
# w are the s allelic frequencies in these regions before dispersal. X′′′′,Y′′′′,
# p′′ and w′′ are these quantities after a life cycle.

| | |
|---|---|
| [X′,Y′,p′,w′] | = NCR_Dispersal(X,Y,p,w,r,z,G); |
| [X′′, Y′′] | = NCR_Birth(X′,Y′,F); |
| [X′′′, Y′′′, p′′, w′′] | = NCR_Selection(X′′,Y′′,p′,w′,L_{T},h_{T},K_{T},L_{R},h_{R},K_{R}); |
| [X′′′′, Y′′′′] | = NCR_Density_Dependence(X′′′,Y′′′,a,b); |

return [X′′′′, Y′′′′, p′′, w′′];

end;
NCR_WinterSurvival(X,Y, μ);
# Reduces population due to winter survival rate μ.

X_{w}= μX;
Y_{w}= μY;
return [X_{w},Y_{w}];

end;
NCR_Dispersal(X,Y,p,w,r, z, G);
# A fraction, 1-r, of the insects do not disperse. Of the remaining insects that do
# disperse, # their choice to land in the transgenic field is dependent on their preference
# for it, z, and the relative size of the refuge, G. Higher preference or a lower refuge size
# or both favor the transgenic field.
#
# X and Y are the insect population densities in the transgenic field and refuge, and p
# and w are the s allelic frequencies in these regions before dispersal. X′,Y′,p′ and w′
# are the same quantities after dispersal.

X′ = (1-r)X + zr(X+Y)/(z+G);
Y′ = (1-r)Y + Gr(X+Y)/(z+G);
p′ = ( p(1-r)X zr(pX + wY)/(z+G) ) / X′
w′ = ( w(1-r)Y Gr(pX + wY)/(z+G) ) / Y′
return [X′, Y′, p′, w′];

end;
NCR_Birth(X′, Y′, F)
# X′ and Y′ are the insect population densities in the transgenic field and refuge
before
# the birth phase. X′′,Y′′ are the same quantities after birth. F is the average adult
# fecundity.

X′′ = F X′;
Y′′ = F Y′;
return [X′′, Y′′];

end;
NCR_Selection(X′′,Y′′,p′,w′, K_{T}, h_{T}, L_{T}, K_{R}, h_{R}, L_{R});
# X′′ and Y′′ are the insect population densities in the transgenic field and refuge,
and
# p′ and w′ are the s allelic frequencies in these regions before selection. X′′′,Y′′′,p′′
# and w′′ are the same quantities after dispersal. Details can be found in Crow and
# Kimura (1970).

X′′′ = (L_{T}p′² + 2(L_{T} + K_{T}(1-h_{T}))p′(1-p′) + K_{T}(1-p′)² ) X′′;
Y′′′ = (L_{R}w′² + 2(L_{R} + K_{R}(1-h_{R}))w′(1-w′) + K_{R}(1-w′)² ) Y′′;
p′′ = (L_{T}p′² + (L_{T}h_{T} + K_{T}(1-h_{T}))p′(1-p′) ) X′′ / X′′′
w′′ = ( L_{R}w′² + (L_{R}h_{R} + K_{R}(1-h_{R}))w′(1-w′) ) Y′′ / Y′′′
return [X′′′, Y′′′, p′′, w′′];

end;
NCR_Density_Dependence(X′′′,Y′′′, a, b);
# DD (Density Dependent Survival) accounts for the limited food supply. A large
# population will suffer higher losses than a small one due to resource depletion
# [Hassell, 1974].

X′′′′ = X′′′ (1 + aX′′′)^{-b};
Y′′′′ = Y′′′ (1 + aY′′′/ G)^{-b};
return [X′′′′, Y′′′′];

end;

In an alternative embodiment, the resistant strain is selected for using an EMS-mutagenized line (Chemical mutagenesis), a P-element mutagenized line (transposable elements), site-directed mutagenesis, gain of function mutant line, loss of function mutant line, or any other mutagenized genomic line selected for pesticide resistance.

Chemical mutagensis is the use of any chemical that increases the mutation rate in an organism or in a population of organisms. Transposable elements are those elements that can transpose themselves throughout the genome. These are mobile elements of DNA that can move their position in the genome. Gain of function mutant line is a line where a gene or hene expression has been altered to confer a new phenotype. This new phenotype has resistance or susceptibility to a toxin. A loss of finction mutant line is an organism or a line where a gene or gene expression has been lost and this confers a new phenotype. This new phenotype has resistance or susceptibility to a toxin.

Generation of Resistant Lines or Alleles

In another aspect of the invention, resistance traits are selected for and are used in the discovery of NCR toxins. A first resistant allele is an allele that confers resistance to an initially delivered toxin. The first resistant allele can refer to many different alleles that will confer resistance to the first toxin or only one of two NCR toxins. A dually resistant allele is an allele that confers resistance to both the first toxin and the second toxin (the second toxin being the negative cross-resistance toxin). In one embodiment, the initial resistance allele is generated against the first toxin with the intent of using the first resistance allele in the discovery of NCR toxins.

In an exemplary embodiment, an organism is made transgenic for a toxin. The organism is mutagenized to generate at least one resistance allele in the target genes. The production of the toxin is then turned on. The toxin will be present internally in the organism and only those organisms with the new resistance gene or allele survive. The resistance gene or allele is then utilized to discover NCR toxins.

In another aspect of the invention, a heterozygous resistant line can be created and used in the discovery of NCR toxins. This will allow for direct screening of toxins that kill the heterozygous organisms without having to screen the homozygous resistant organisms.

In an exemplary embodiment, an organism is made transgenic for both the susceptible and resistant alleles, such that all progeny will be heterozygous for the resistance and susceptible alleles. In Drosophila melanogaster one can make the insect transgenic for example using a P element vector system such as pCaSpeR or pCaSpeR-Hs (FIG. 14A) where the transgenes can be the target site for the first toxin. The Drosophila can be made transgenic with the resistance or susceptible allele or both. In Drosophila one can transform the two resistant and susceptible alleles into separate balancer chromosomes that will allow one to establish a fly line that can only be heterozygous for resistance. Balancers are chromosomes that are designed to maintain a homologous chromosome or a segment of a homologous chromosome intact in a stock; they are usually lethal in the homozygous condition. But some balancers can exist in the presence of other balancers for the same or substantially the same chromosome. For example in Drosophila CyO, Curly of Oster, and TM6 are both second chromosome balancers that can exist together. A fly stock containing, CyO and TM6 will always be heterozygous for CyO/TM6, since CyO over CyO and TM6 over TM6 are not viable. Thus, if a resistance allele was transformed onto one balancer, for example CyO, and the susceptible allele was transformed onto TM6, one could guarantee a heterozygous stock of Drosophila. This heterozygous stock could be used in screens for NCR compounds. Such a system for maintaining a heterozygous stock for use in negative cross resistance screens is not limited to use of balancers, transgenics, or Drosophila melanogaster.

### Screening for NCR in Toxins No Longer Effective Against Susceptible Insects

In another aspect of the invention, an initial screen is performed that determines compounds less effective against susceptible lines. These compounds are then tested for NCR activity to the lines considered resistant.

For example, a transgenic fly (known to be susceptible to toxin A) is generated to include toxin A. The toxin within the fly is mutagenized (at some point in the process) instead of the fly. In one embodiment, the toxin is altered either before transforming the flies or after transforming the flies and then the flies are scored for toxins no longer toxic to the susceptible flies. In an exemplary embodiment, the transgenic flies are heat shocked. The transgenic toxin is under a heat shock promoter and thus the toxin is produced. Any of the toxins still toxic to the insect will kill the insects. Thus a population of susceptible flies is created that contain a toxin no longer toxic to the susceptible flies. In parallel experiments resistant lines can be generated that are resistant to the original toxin A. The R/R (homozygous resistant to toxin A) are crossed with the S/S (homozygous susceptible to toxin A) containing mutagenized toxin A (which no longer kills S/S). R/R or R/S flies lines are then established with the mutagenized toxins. A fly line R/S with a mutagenized toxin A has thus been created. At least some of the insects are heat shocked from each line to determine whether high mortality exists. If the mutagenized toxin shows useful NCR activity, it will kill the R/S and/or R/R flies. It is believed that the above strategy is beneficial when working with novel transgenic technologies.

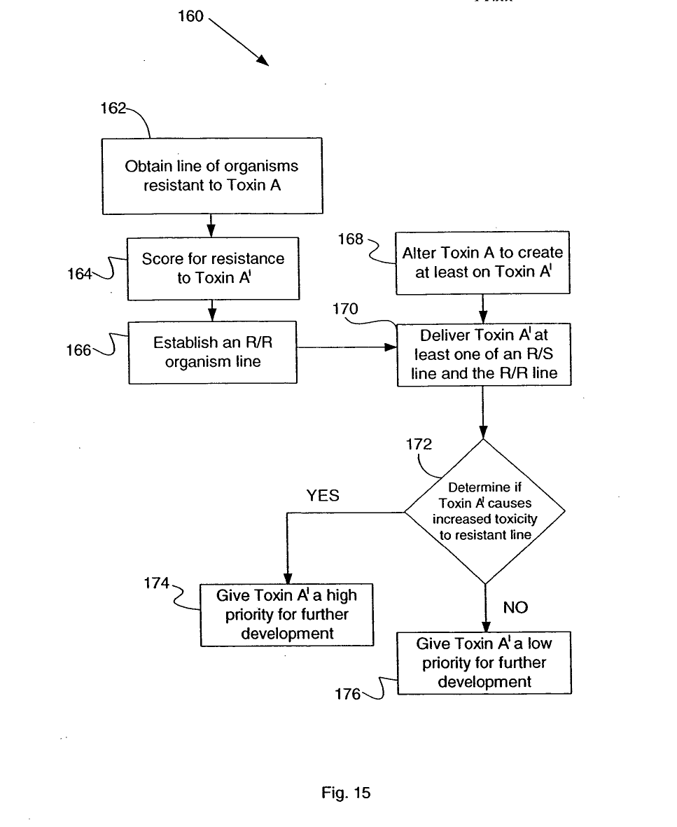

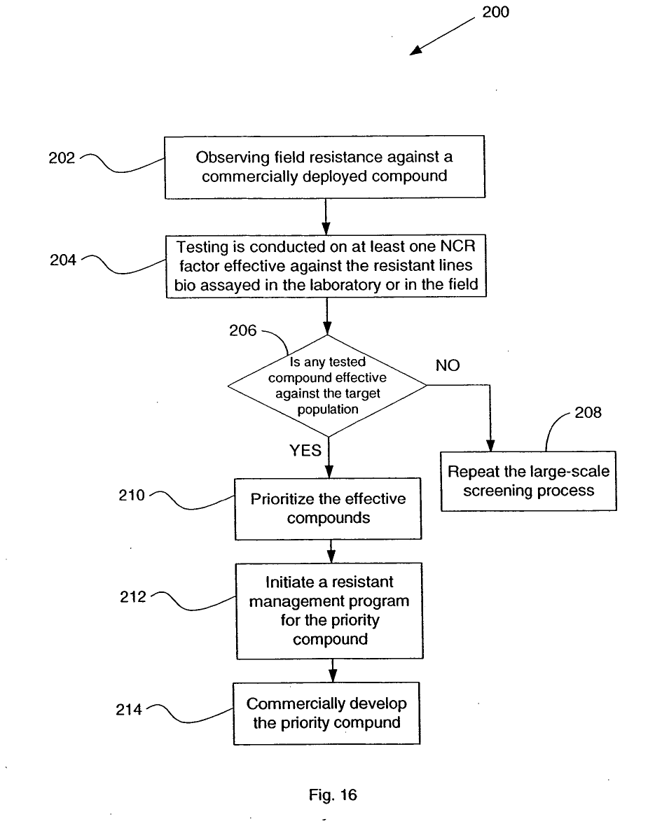

A screening method 160 is illustrated in FIG. 15. Method 160 includes obtaining 162 a line of organisms, e.g., flies, resistant to a first toxin, such as Toxin A. The organisms are scored 164 for resistance to Toxin A and an R/R organism line is established 166. In addition, Toxin A is altered 168, such as by mutation, to generate at least one Toxin A′. Toxin A′ is delivered 170 to at least one of the R/R line or an R/S line of the organism. It is then determined 172 whether Toxin A′ causes increased toxicity to either or both of the R/R line and the R/S line. If Toxin A′ causes increased toxicity to one of the resistant lines, Toxin A′ is given a high priority 174 for further development. If Toxin A′ does not cause increased toxicity to one of the resistant lines, Toxin A′ is given a low priority for further development.

In an illustrative example, a para^{tsl} fly line is defined only as susceptible to deltamethrin (TOXIN A) and a Canton-S fly line is, relatively speaking, a line resistant to deltamethrin. The para^{tsl} line is treated with a plurality of compounds to determine whether one or more of the compounds have a relatively diminished ability to kill para^{tsl}. This diminished ability is shown with bioassay results (See for example, Pittendrigh et al., 1997 paper on the para^{ts} mutants). The compounds with a diminished ability to kill para^{tsl} are then tested against either or both of the R/S and R/R organisms. This approach provides a NCR toxin that has the minimal toxicity to the susceptible insects which makes for a much more powerful use of an NCR toxin pair in an active refuge system, and elsewhere. Thus the order of evaluations for the NCR factors is actually reversed from conventional methods, which is beneficial when using recombinant technology and proteinacious toxins to develop NCR toxins.

FIG. 14A is a method 200 of providing management decisions for the development of NCR factors. Method 200 includes observing 202 field resistance against a compound commercially deployed to reduce the number of pests in a target population on a tract of land. Testing 204 is conducted on a group of NCR factors that were discovered to be effective against the resistant lines bio-assayed in the laboratory. The testing is conducted against the field resistant line of the target population. A determination is made 206 regarding whether any compound tested is effective against the target population. If no compounds are found to be effective against the target population, the large scale screening process is repeated 208 using field resistant pests in the bioassay. If compounds are found to be effective against the target population, the effective compounds are prioritized 210 and a decision is made for each such compound regarding whether to initiate 212 a resistant management program to slow entry of the resistant alleles into the pest population and commercially develop 214 the compound.

A first factor influencing the decision to develop a NCR factor is the diversity of resistance mechanisms in the field. If the forms of resistance that occur in the field are highly uniform (i.e., similar forms of resistance) this uniformity increases the priority given to the development of negative-cross resistance factors. Commercial development of a NCR factor may be feasible if there are highly uniform amino acid changes across divergent taxa and the resistance mechanism is uniform in the field. However, if there is a great diversity in nature regarding how pests develop resistance to a commercially deployed pesticide, it may be difficult to identify a single compound that provides generalized NCR. If the resistance shows diversity, development of a single NCR factor may be expensive and multiple NCR compounds may prove too costly for combating resistance.

Economics is the second factor influencing the decision to develop a NCR factor. Resistance to toxins that have little commercial value, such as insecticides that are useful in minor or niche markets, may not justify the costs of developing NCR factors. Alternatively, resistance to commonly used antibiotics may warrant the development of multiple NCR factors (effective against different forms of resistance) due to the commercial value of these compounds.

Screens for NCR factors may also be useful in the discovery of compounds that kill antibiotic resistant bacteria. Large-scale screens can be performed with antibiotic susceptible and resistant bacterial strains to identify compounds that selectively kill the antibiotic resistant (but not the susceptible) bacteria. These compounds can then be incorporated into materials or medicines that reduce the numbers of antibiotic resistant bacteria in a given environment. As with pesticide resistance, the diversity of resistance alleles and the nature of the antibiotic resistance genes influence how feasible such a screen is for dealing with antibiotic resistance.

For example, after performing method 100 (shown in FIG. 6) on a compound, and prior to commercial development of the compound, testing is conducted to determine how useful the compound would be in the field. Field trials of the compound include testing a field including a first organism, e.g., an insect, a plant, a bacteria, a virus, a fungus and others, with a mixture of resistant and susceptible lines (genotypes/phenotypes). The frequency of resistance is determined before application and after application to determine an effectiveness of the compounds. Thus, the above described methods provide for screening a compound, such as in a lab, determining a potentially beneficial compound, and testing the potentially beneficial compound in the field.

Implementation of Negative Cross-Resistance Factors

The development and effective deployment of NCR factors utilize a combination of basic research, marketing, extension services, and field applicators of the product. In one embodiment, research teams access field collected lines of resistant pests to develop effective toxins capable of killing pests that contain those alleles thought most likely to emerge in a field population. Once basic research programs determine the underlying molecular basis of field resistance this information is used to develop molecular diagnostics to determine the frequency of the allele in the pest population being targeted.

Marketing programs along with extension services provide feedback on the allelic frequency of resistance in the field. Decisions regarding when to begin to use the second NCR factor are made once the allelic frequency of resistance in the field is known. If monitoring programs are too costly or ineffective, treatment of the target population early in the growing season to reduce the resistance allele in the population is sometimes an effective way to minimize the development of a resistant pest population throughout the field season. In one embodiment, the NCR factors are delivered to the target population utilizing at least one of sprays, pellets, powders, pills, vaccines, gels, creams, syrups, gel tablets, sub-cutaneous injections, liquids, suspensions, food products, baited or non-baited traps, and transgenic organisms.

In another embodiment, the NCR factor is sold with the first toxin in the same or substantially the same or separate product or products. The NCR toxin is used at tie end of the patent life of the first toxin to sell or deliver a product to the market place that allows the company to maintain a proprietary toxin system in the market place.

### EXAMPLES

A model for the developmental cycle of a hypothetical insect pest was formulated and the following assumptions were made. The number of progeny per female had a Poisson distribution, mean fecundity, μ and was assumed to be 60 offspring per female. When there were fewer than 20 mating pairs the progeny was simulated directly. Above 20 mating pairs, the total number of progeny was approximated using the normal distribution. The number of insects of each genotype among the total progeny has a multinomial distribution with the proportion of each genotype being given by their respective Hardy-Weinberg frequencies. The number of males and females was binomially distributed with the expected ratio of males to females in the population being 1:1. The proportion of mutant alleles in the population is initially equal to m (mutation rate). Initially there are Nμ insects in different stages of development in the population. Each insect mates once on average. Mating occurs, with equal probability, at any time during the breeding period of the insect's adulthood (3.5 days of breeding). Mutation rate (per individual, per unit time>is constant, as is female fecundity. The developmental times for the following stages are egg (7 days), larval development (30 days) and reproductive period (3.5 days). The standard (background) mortality rate was calculated using an egg mortality of 5%, a larval establishment mortality of 5%, and a larval developmental mortality of 20%. It is flirther assumed that the genotype does not affect the background mortality rate such that the background mortality is independent of the mortality caused by the pesticide or pesticides.

A Monte Carlo (which randomly generates values for uncertain variables over and over to simulate a model) simulation tool, i.e., a decisioneering tool, was used to estimate the maximum population size and mutant allelic frequency after a given number of days. The model calculated these values at each half-day. Given the number of adults mating during a time interval, the number of progeny was determined, and these progeny were classified as homozygous wild-type, homozygous mutants, or heterozygous for these alleles (based on Hardy-Weinberg frequencies). Selection and mutation occurred before the mating phase for each insect.

Simulation refers to any analytical method with the intent to imitate a real-life system. This is especially useful when other analyses are too mathematically complex or simply too difficult to reproduce in a laboratory or field situation. One type of simulation is the Monte Carlo simulation, which randomly generates values for uncertain variables over and over to simulate a model. This gives one variability in the model allowing for extremes in outcome as opposed to a deterministic model that gives the average of what will occur when the experiment is repeated infinite times. The Monte Carlo simulation is a strategy to emulate the variability that can occur in a biological system.

The definition of Hardy-Weinberg frequencies is as follows. The shuffling of two alleles of one gene through segregation during meiosis and recombination during fertilization do not cause a change in the frequency of these two alleles. Since gene frequencies will then remain stable after we perform our selection step in the model the frequencies of diploid zygotes (the next generation of insects) can be predicted from them by using the statistical theorem: p\hat{ }2(RR)+2pq (RS)+q\hat{ }2(SS)=1.

As expected, if the two toxins did not cause equal mortality rates to the respective homozygous insects, the alleles of the fitter homozygotes became more common over time. In all but one of the cases examined, the population size was below the starting level of 10,000 individuals when the fitter allele reached a frequency of 50%. As the allelic frequency of the fitter allele increased beyond 50% the population grew rapidly. The closer the two NCR compounds were in toxicity, the longer it took for the fitter allele to increase in frequency. High toxicity of both compounds to homozygotes and heterozygotes resulted in effective control of the insect population.

When the combined toxins impacted the heterozygotes less than the homozygotes, the allelic frequency of the resistance allele approached 50% asympotically. The greater the difference in mortality rates between the heterozygotes and homozygotes the faster the resistance allele approached 50% frequency. But if the combined toxins killed more heterozygotes than homozygotes and the resistance allele was rare at the start of the experiment, the allele was likely to remain rare.

If the resistance allele was already common in the population when the two-toxin regime (both compounds used at once) was initiated (where there was greater heterozygote toxicity than homozygote toxicity), the resistance allele ultimately went to an extreme value (near 0% or near 100%). The probability of the allele going to fixation (100%) or extinction (0%), or close to these values, depended on its starting frequency. If the resistance allele started below 50%, on average it would tend towards extinction. The further the starting allelic frequency was below 50% the greater the probability that the allele would be lost from the population. On the other hand, if the frequency of the resistance allele exceeded 50% at the start of the concurrent treatments, then the resistance allele would on average go to fixation. If the starting allelic frequency was at 50% then the allele was as likely to tend towards fixation as it was to go to extinction.

Simulations were also performed in which both NCR factors were applied together and their toxicity was varied to determine which conditions provided for the most effective control of the insect population. Four specific cases were investigated. First, the mortality rate of one homozygote group was 99% and the mortality rates of the heterozygotes and the other group of homozygotes was varied. Second, the effect of varying heterozygous fitness was examined when the mortality rates of the two homozygous groups were equal. Third, the impact of starting allelic frequency on the fate of the alleles was reviewed when the two toxins killed the heterozygotes at a higher rate than the homozygotes. Fourth, the second toxin was used intermittently to minimize the frequency of the resistance allele.

### EXAMPLE

A test was conducted utilizing an initial screen of DDT and 8 pyrethroids against Canton-S (DDT susceptible flies) and para^{tsl} (DDT resistant flies). Control flies, flies not exposed to toxins, were also screened and showed no mortality. Table 6 illustrates the results of this initial screen.

TABLE 6

| | Fly Line Preferentially | Experiment-wise |
|---|---|---|
| Compound | Killed by the Toxin | P-Value |
| DDT | Canton-S | 0.039 |
| Deltamethrin | Para^{tsl} | 0.0156 |
| D-trans allethrin | Neither | >0.20 |
| Tempo | Para^{tsl} | 0.125 |
| Asana | Neither | >0.20 |
| Resmethrin | Para^{tsl} | >0.20 |
| PP321 | Neither | >0.20 |
| Permethrin | Para^{tsl} | 0.039 |
| PP993 | Canton-S | 0.18 |

As seen in Table 6, across the doses, deltamethrin and permethrin were more toxic to para^{tsl} flies than to Canton-S flies.

Table 7 illustrates the mortality of Canton-S flies and para^{tsl} flies in the presence of either DDT or deltamethrin. Control flies showed no mortality.

TABLE 7

| | | Percent Dead | | P-Values | |
|---|---|---|---|---|---|
| Compound | Dose | Cantons-S | Para^{tsl} | Non-Corrected | Corrected |
| DDT | 20 | 71.7 | 6.7 | 0.012 | 0.084 |
| | 15 | 75.0 | 8.3 | 0.0059 | 0.041 |
| | 10 | 43.3 | 13.3 | * | * |
| | 8 | 48.3 | 23.3 | * | * |
| | 5 | 35.0 | 6.7 | * | * |
| | 2 | 15.0 | 3.3 | * | * |
| | 1 | 6.7 | 0 | * | * |
| | 0.5 | 8.3 | 0 | * | * |
| | 0.2 | 11.7 | 13.3 | * | * |
| Deltamethrin | 20 | 98.3 | 100 | * | * |
| | 15 | 100 | 100 | * | * |
| | 10 | 98.3 | 98.3 | * | * |
| | 8 | 96.7 | 100 | * | * |
| | 5 | 100 | 100 | * | * |
| | 2 | 93.3 | 100 | * | * |
| | 1 | 26.7 | 85.0 | 0.0128 | 0.102 |
| | 0.5 | 56.7 | 100 | * | * |
| | 0.2 | 31.7 | 98.3 | 0.0037 | 0.030 |

*P > 0.20

As illustrated in Table 7, when tested by individual doses, only deltamethrin preferentially killed para^{tsl} flies. The other pyrethroids tested did not show significantly different toxicity between Canton-S flies and para^{tsl} flies at any of the specific doses.

As a further step in the test, heterozygotes were tested with either deltamethrin or DDT. The effect of deltamethrin on Canton-S flies, para^{tsl} flies, and heterozygous fly lines is illustrated in Table 8.

TABLE 8

| | | | | | Resistance |
|---|---|---|---|---|---|
| Fly Line | Male/Female | LD₅₀ | 95% CI | N | Ratio |
| Canton-S | Female | 0.988 | 0.075-2.22 | 830 | 24.7 |
| | Male | 0.396 | 0.287-0.495 | 420 | 9.9 |
| Para^{tsl} | Female | 0.057 | 0.043-0.074 | 500 | 1.4 |
| | Male | 0.040 | 0.025-0.057 | 520 | 1.0* |
| Heterozygotes | Female | 0.860 | 0.652-1.128 | 830 | 21.5 |

*The resistance ratio is defined as the respective fly line LD₅₀ as the numerator and the LD₅₀ of the para^{tsl} males as the denominator.

As seen in Table 8, heterozygotes are resistant to deltamethrin and resistance is dominant. In addition, it is known that heterozygotes are resistant to DDT. Thus it was determined that resistance was not recessive to either deltamethrin or DDT. An additional test was conducted to determine if DDT and deltamethrin applied at the same time killed the heterozygotes.

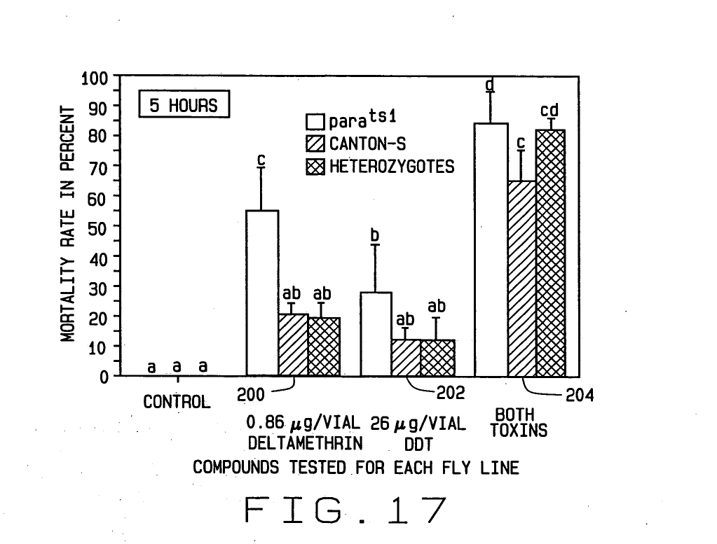

FIG. 17 graphically illustrates the application of deltamethrin by itself 220, the application of DDT by itself 222, and the application of both deltamethrin and DDT at the same time 224 to para^{tsl} flies, Canton-S flies, and heterozygotes. As illustrated in FIG. 17, both deltamethrin and DDT together killed the heterozygotes better than either deltamethrin or DDT on its own. Therefore, DDT and deltamethrin combined effectively to kill the heterozygotes and it was determined that deltamethrin and DDT are NCR factors.

In another embodiment, NCR toxins are selected for and tested to determine their strength. These initial NCR toxins are then used as a starting material for evolutionary techniques, as described below in greater detail, to identify or create a strengthened NCR toxin or toxins. Exemplary evolutionary techniques include, but are not limited to, phage-display technology, any in vitro mutagenesis technique to alter proteins and then selection of toxins that are more toxic to homozygous or heterozygous resistant organisms than the original protein, biopanning selection of enriched phage populations, cell-display technologies for rapid evolution of proteins, directed mutagenesis for known key residues to increase NCR activity, high affinity recombinant antibody fragments, yeast technologies with the same or substantially the same intent as the phage-display technology, DNA shuffling, and combinatorial chemistry.

These strengthened NCR toxins are evolved using in vivo or in vitro selection techniques to achieve greater NCR activity. A strengthened NCR toxin is defined as an evolved toxin that is more toxic to resistant (homozygous, heterozygous, or both) pests than the initial NCR toxin. For example, the initial NCR toxin, or first toxin, is one of a weak toxin, a moderate toxin, and a strong toxin. Exemplary compounds and potential target sites include, but are not limited to, Bacillus thuringiensis toxins and derivatives thereof, Photorhabdus luminescens protein toxin and derivatives thereof, Xenorhabdus nematophilus protein toxin and derivatives thereof, insecticides, naturally-occurring plant defense proteins, herbicides, nematacides, fungicides, and hormones and plant growth regulators.

In one embodiment, the initial toxins are discovered through NCR screens as described above. In an alternative embodiment, the initial toxins are known NCR toxins. In an exemplary embodiment, the first toxin has a toxicity as indicated below.
(i) 1.1 to 10-fold greater toxicity to resistant than to susceptible,
(ii) 10 to 20-fold greater toxicity to resistant than to susceptible,
(iii) 20 to 50-fold greater toxicity to resistant than to susceptible,
(iv) 50 to 1 toxicity to resistant than to susceptible,
(v) 100 to 1000-fold greater toxicity to resistant than to susceptible,
vi) 1000 to 10,000-fold greater toxicity to resistant than to susceptible, and
vii) 10,000 to 1,000,000-fold greater toxicity to resistant than to susceptible.

Once the initial toxin is determined, the toxin is developed into a strengthened toxin through, for example, rapid evolution of the toxin and selection for specificity to a resistant target site. Resistant is defined as an altered target site that confers resistance to a first compound group such as an insecticide, herbicide, fungicide, or antibiotic or that is effective against killing organisms that have developed resistance to a first viral form. The toxicity of the altered toxin is then determined in a screen against homozygous and/or heterozygous resistant organisms to test for greater toxicity as compared to the initial toxin used as the starting material.

For example, an initial NCR toxin is discovered through a NCR screen, as described above, that is 1.5-times more toxic to a resistant pest than to a susceptible pest. Phage-display technology is then utilized on this toxin to increase the ratio of toxic to resistant as compared to susceptible pests. This increase is between 1.6 times to 10,000,000 times greater toxicity to the resistant as compared with the susceptible. The strengthened NCR toxin is re-evaluated against homozygous and/or heterozygous pests to determine and select the most effective form of the toxin. The selected NCR toxin is then deployed as one of an insecticide, a herbicide, an antibiotic, a fungicide, and a nematode.

The following is an exemplary embodiment of selectively increasing the toxicity of molecules. The example utilizes phage-display technology to selectively increase the toxicity of molecules. Cysteine proteinase inhibitors (cystatins) from plants have been implicated as molecules of defense against insects, such as Coleopteran and Hemipteran insects. Koiwa et al. (1998) investigated two soybean cystatins to determine if phage display technology could be used to select out forms of an inhibitor or insecticidal protein from proteins with lesser insecticidal activity. Two cystatins were utilized in their investigation, soyastatin N (scN) and soystatin L (scL), which have 70% sequence identity between the two proteins. ScN is a much more potent inhibitor of vicilin peptidohydrolase, papain, and insect gut proteinases.

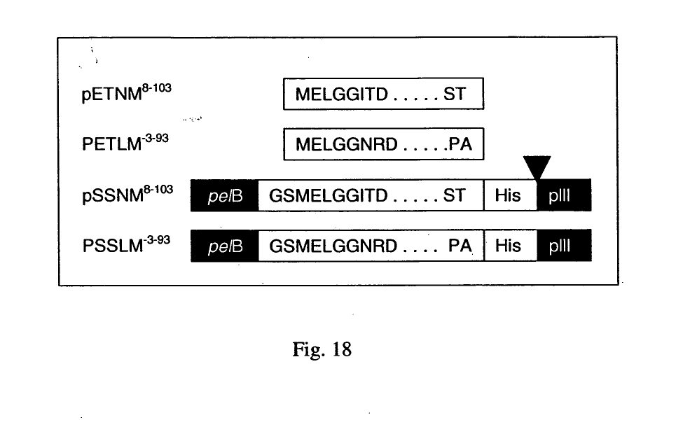

FIG. 18 illustrates amino sequences of soluble and phage display recombinant soyacystatin proteins (Kiowa et al. 1998). The N— and C-terminal amino acids are shown and the dots are representative of the intervening sequence of scL and scN (Botella et al., 1996). The cDNA open reading frames of soyacystatins were introduced into pET28a to produce the resultant pETNM8-103 and pETLM-3-93 for expression of soluble (untagged) scNM8-103 or scLM-3-93 (open box). The phage display soyacystatin proteins were expressed as fusions with the pelB leader sequence (pelB), 6×His tag (His) and plll protein from vectors where the cDNA inserts were sub-cloned into pSSHisA to produce pSSNM8-103 or PSSLM-3-93. The arrowhead indicates the position of the glutamic acid encoded by the stop codon.

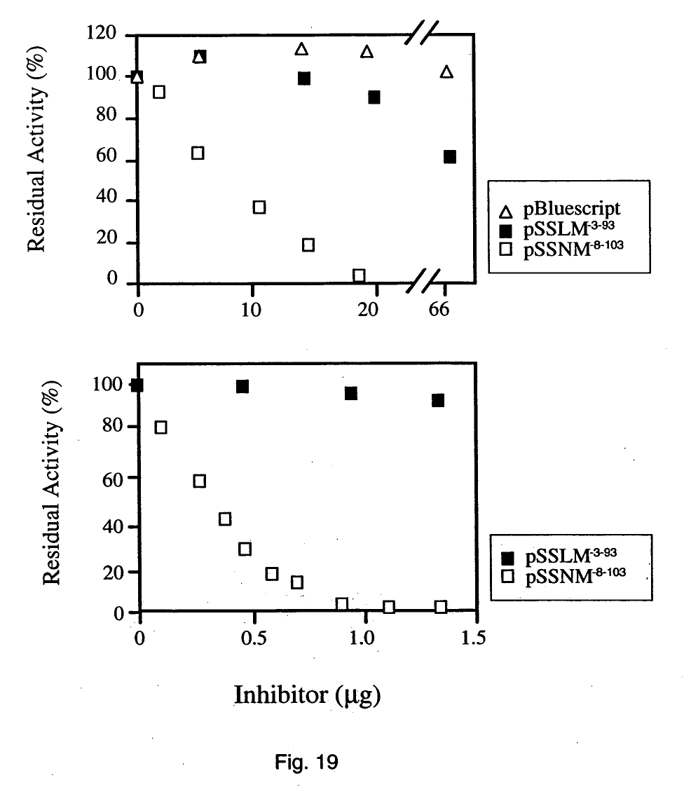

FIG. 19 illustrates that the differential papain inhibitory activity of soyacystatins is analogous between phage-displayed and soluble recombinant proteins (Kiowa et al. 1998). During the experiment, pre-activated papain was incubated with N-benzoyl-DL-arginine-β-naphthylamide to establish 100 % activity. Each illustrated point is the average of five replicate samples. (A) Phage particles and (B) soluble recombinant proteins. Both cystatins were displayed on phage particles and then selected using papain-binding affinity. The scN protein showed much higher affinity to papain than did scLM.

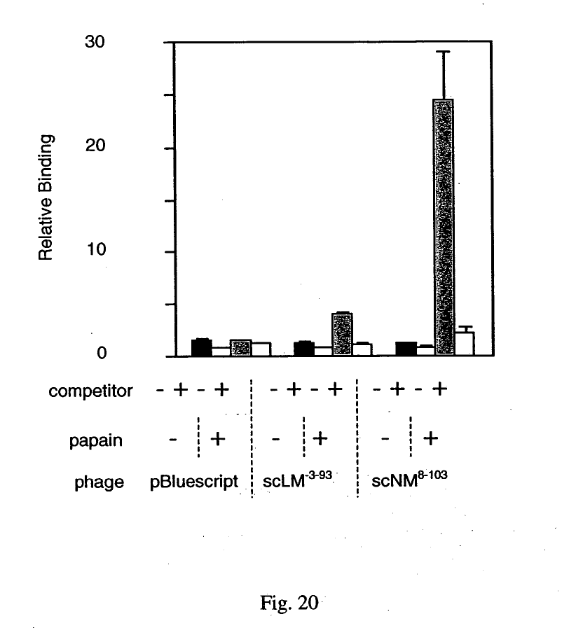

FIG. 20 illustrates a representative experiment of biopanning selection of phage-displayed soyacystatins (Kiowa et al., 1998). Each well of a microtiter plate was coated with a solution of PBS without (−) or with (+) 30 μg m⁻¹ of papain. Phage particles were purified from bacteria harboring pBluescript, pSSLM⁻³⁻⁹³ (scLM⁻³⁻⁹³) or pSSNM⁸⁻¹⁰³ (scNM⁸⁻¹⁰³) and the binding reaction was performed in the absence (−) or presence (+) of chicken cystatin (competitor). A number of bound phage particles was determined as XL-1 blue CFU and normalized to CFU of the papain/competitor treatment for each phage preparation. Bars indicate standard deviations. Papain biopanning selection differentially enriched the pool of phage particles expressing scNM molecules. This enrichment resulted in a 200-1000-fold greater enrichment of scN relative to scL.

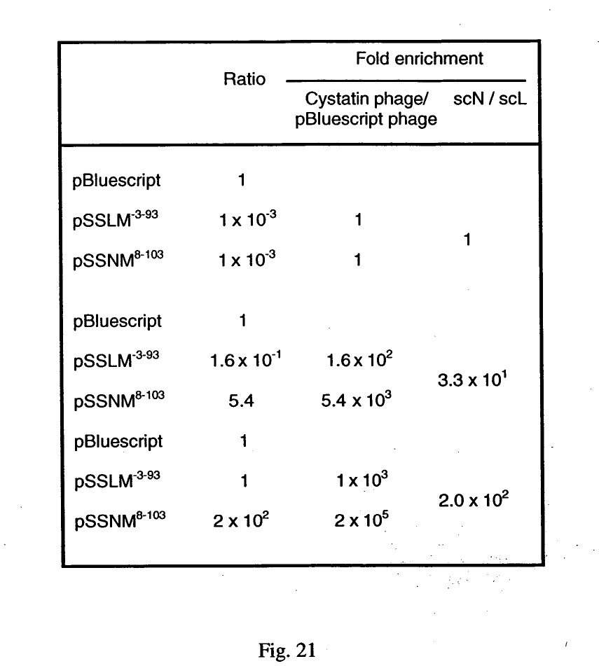

FIG. 21 illustrates Papain biopanning selection differentially enriched for scNM⁸⁻¹⁰³ phage particles to a substantially greater extent than for scLN⁻³⁻⁹³ phage particles. The initial mixture (0) contained the following ratio of phage particles—pBluescript:pSSLM⁻³⁻⁹³:pSSNM⁸⁻¹⁰³=10⁹:10⁶:10⁶ CFU. After the first (1) and second (2) rounds of biopanning selection, bacteria (XL-1 blue) were infected with the eluted phage particles to amplify the selected population. Then the JM105 cells were infected with an aliquot of the population to produce about 400-450 colonies plate⁻¹ w/IPTG+X-gal), i.e. pbluescript colonies were blue (X-gal) and the cystatin (scN or scL)-containing colonies were white. Colony hybridization with an oligonucleotide probe (GGTCTAGAAACATGACTG) specific for a unique region of scN cDNA facilitated detection of pSSNM⁸⁻¹⁰¹ from pSSLM⁻³⁻⁹³ amongst the white colonies (hybridization with scN). The bottom row illustrates hybridization of the scN-specific oligonucleotide probe to an equivalent number of pbluescript, pSSLM⁻¹⁻⁹³ or pSSNM⁸⁻¹⁰³ colonies. After three rounds of biopannng, only pSSNM⁸⁻¹⁰³ colonies were detected.

Table 9 shows an enrichment of soyastatin phage, through biopanning, using an affinity binding technique (Kowa et al., 1998). Affinity binding occurred to papain-coated wells.

TABLE 9

| | | | | Cystatin phage/ | | | | |
|---|---|---|---|---|---|---|---|---|
| | | Total CFU | | pBluescript phage | | Fold enrichment• | | |
| (a) | Phage | scN* | scL° | scN | scL | scN | scL | scN/scL |
| Original | Cystatin | 1.0 × 10⁶ | 1.0 × 10⁶ | 1.0 × 10⁻³ | 1.0 × 10⁻³ | 1 | 1 | 1 |
| Mixture | pBluescript | 1.0 × 10⁹ | 1.0 × 10⁹ | | | | | |
| After 1^{st} | Cystatin | 9.9 × 10² | 9.0 × 10¹ | 8.5 × 10⁻ | 2.8 × 10⁻² | 9.0 × 10 | 8.5 × 10⁻ | 2.8 × 10⁻² |
| Panning | pBluescript | 1.2 × 10³ | 3.3 × 10³ | | | | | |
| After 2^{nd} | Cystatin | 9.9 × 10² | 9.0 × 10¹ | 8.5 × 10⁻ | 2.8 × 10⁻² | | | |
| Panning | pBluescript | 1.2 × 10³ | 3.3 × 10³ | | | | | |

*Original mixture contains scNM⁻⁸⁻¹⁰³ phage and pBluescript phage.
°Original mixture contains scLM⁻³⁻⁹³ phage and pBluescript phage.
•Fold enrichment was calculated by dividing cystatin phage/pBluescript phage ratio of each step by that of the original mixture.

The scN recombinant proteins were then over-expressed in Escherichia coli and incorporated into the cowpea weevil's, Callosobruchus maculates (F.), diet and mortality rates and developmental times of the immature insects (egg to adult eclosion) were assessed. The scN (scNM) protein showed greater mortality rates and slowed developmental time in the cowpea weevil as compared to the scL (scLM) proteins.

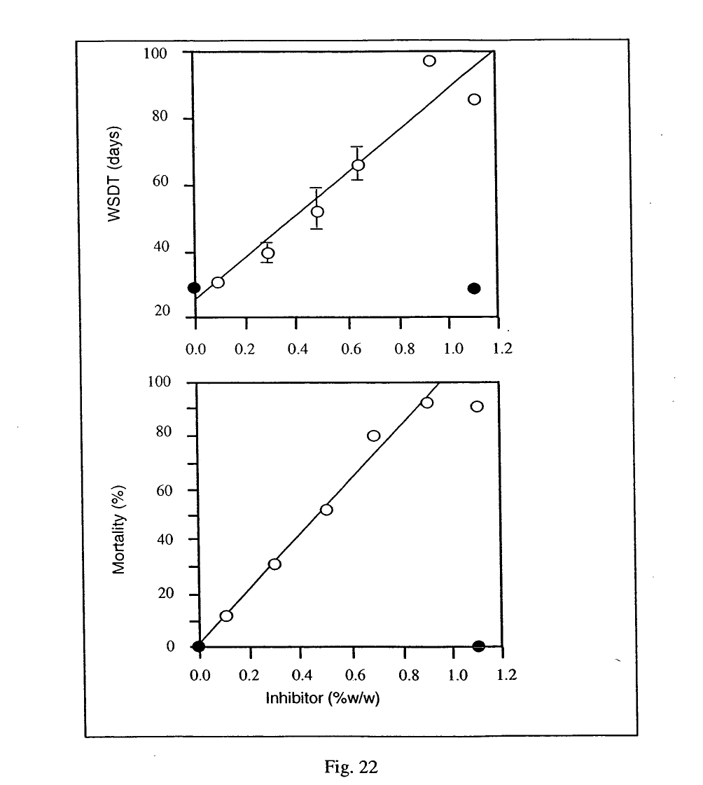

FIG. 22 illustrates that the scNM⁸⁻¹⁰³ protein (O) substantially inhibited cowpea weevil growth and development and resulted in high insect mortality, whereas the scLM⁻³⁻⁹³ protein (●) was relatively inactive (Kiowa et al., 1998). An individual insect egg was placed onto an artificial seed (Shade et. al., 1990) containing no or increasing concentration of soyacystatins. There were 10 seeds per treatment. Within-seed developmental time (upper figure) was determined as a period between when the eggs hatched and when adult insects emerged. Bars indicate standard deviation. Insect mortality (lower panel) was calculated, as a percentage of total eggs placed on seeds, from the number of insects that failed to emerge from artificial seeds. The results of the experiment as illustrated in FIGS. 18-22 and Table 9 demonstrate that proteins can be selectively identified that are more effective in controlling pests, e.g., insects, based on a known target site. Such a target site can be one that is associated with NCR.

The following is an example of selecting for increased biological activity using naive and partially randomized peptide phage libraries of peptide inhibitors of human Factor VIIa (FVII). The VIIa molecule binds with a tissue factor (TF) to form a TF-FVIIa complex that is key to producing enough thrombin to initiate coagulation. One way to inhibit coagulation is to block the interactions between TF and FVIIa. Monoclonal antibodies have been shown to be effective in reducing the TF and FVIIa interaction. As well inhibitors have been shown to reduce the enzymatic activity of the TF-FVIIa complex. Recently, a new series of peptides selected by phage display for binding to the TF-FVIIa complex have been described that result in prolonged TF-dependent coagulation. Peptides were identified and matured from naive and partially randomized peptide phage libraries, using selection against an immobilized Factor VIIa.

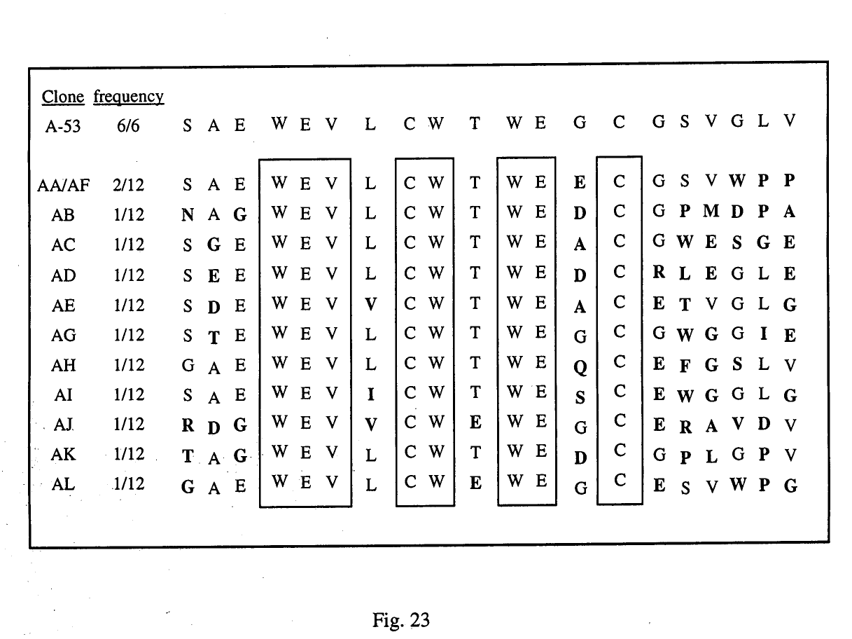

First, peptides were identified and matured that bound to TF-FVIIa. This identification and maturation was accomplished by preparation of a polyvalent phage library of na\dot{i}ve 20-residue peptides, which were diplayed on the major coat protein (P8) of M13. These peptides were sorted against the TF-FVIIa complex (hard randomization). The pool was enriched by 1500-fold. Six representative clones were selected and sequenced. These encoded for a peptide, known as A-53, (see for example, Dennis et al. (2001). (FIG. 23)

FIG. 23 illustrates the identification of proteins that have affinity to a target, in this case FVIIa and illustrates sequences of Phage Clones Selected from Na\dot{i}ve and Partially Randomized Libraries for Binding to TF-FVIIa^{a}. In FIG. 23, sequence A-53 was identified from the naive peptide library, X₇CX₅CX₆. Residues completely conserved during soft randomization are boxed. New amino acid residues that appeared during soft randomization are in bold. Sequences AA through AL were identified during soft randomization of A-53. Clones AA and AF share the same DNA sequence. Peptide A-57 was derived from the sequence in clone AD (21).

A-53 was then affinity matured using “soft randomization” methodology. This approach started with the A-53 peptide sequence and then introduced a mutation rate of 50 percent at each position. This oligonucleotide was then used to generate a peptide library that was fused to the P3 protein, which is a minor protein of M13. This partially randomized library included approximately 2×10⁷ clones and was then sorted against immobilized TF-FVIIa. Four rounds of selection were conducted and an enrichment of 10⁵ and achieved. Clones were then sequenced and it was found that eight amino acid positions were invariant.

A final monovalent peptide library was produced, in which these eight positions were fixed and the remaining amino acid positions were randomized (other 12 positions were varied). Four more rounds of selection occurred with another enrichment of 10⁵. Then, twenty-three clones were sequenced to calculate the amino acid preferences at the remaining positions to develop a consensus sequence, designated as ‘C’ (MEEWEVLCWTWETCERGEGQ).

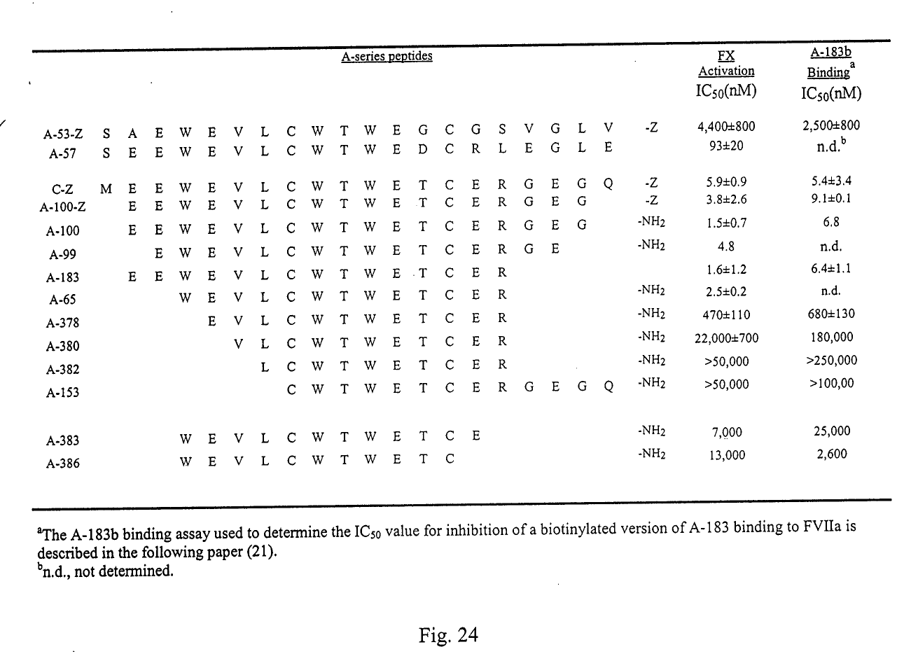

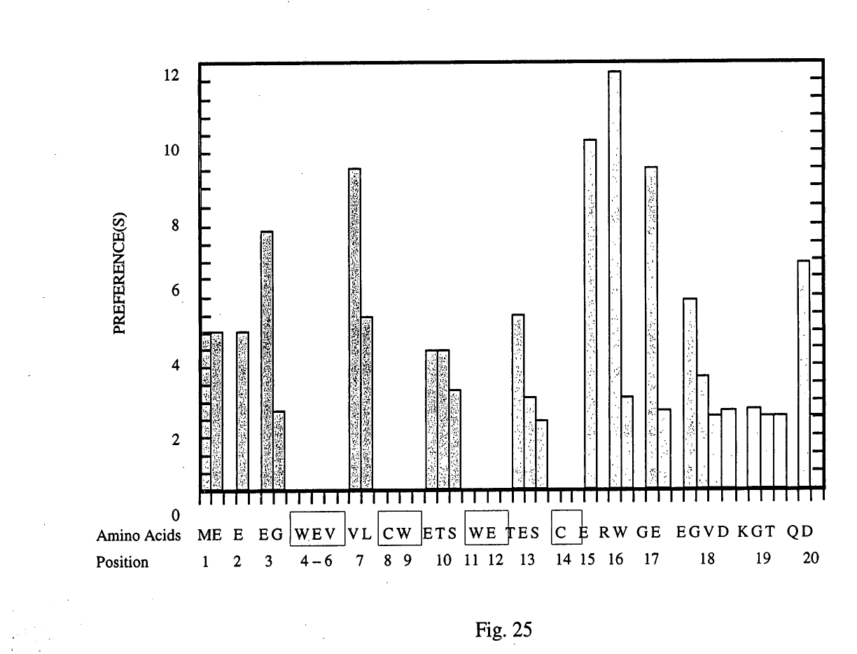

FIG. 24 illustrates the inhibition of Factor X Activation and A-183-b binding by A-Series peptides. FIG. 25 illustrates sequence preferences using full randomization. The library X₃WEVXCWXWEXCX₆, where amino acids in bold were held constant and all 20 amino acids were substituted at X, was sorted against TF-FVIIa. Amino acids identified at the indicated randomization positions are plotted as a function of their preference. The preference for any amino acid is reported as the number of standard deviation units above a random chance occurrence of a given residue assuming binomial distribution and accounts for codon bias and sampling statistics. Fixed amino acids in the library were boxed.

Peptides were then produced, using a recombinant methodology, and purified. The purified peptides had their non-crucial (for binding) regions truncated, resulting in a 15-residue peptide, which was then fused to a “Z-domain” useful as an affinity handle for purification. This fusion peptide was then purified. An inhibition of FX activation study of fusing the peptides was performed. The molecules tested were as follows: (1) the initial lead compound, A-53-Z, from the naive library; (2) the consensus sequence from the hard randomization, C-Z; and A-100-Z which resulted from the final soft randomization of the final consensus sequence. The results are shown in Table 10 and included greater inhibition of FX activation at each stage of evolution of the inhibitor.

Peptides is defined as any group of various amides that are derived from two or more amino acids by combining the amino group of one acid with the carboxyl group of another. Peptides can also be obtained by partial hydrolysis of proteins. Recombinant is relating to or containing recombinant DNA. The DNA is typically, but limited to, DNA that has been altered by a technology or a biotechnology. Purified is defined as to make pure. The “Z-domain” is useful as an affinity handle for purification. An affinity handle for purification is defined as a region that allows one to temporarily attach a given molecule to a solid, such that molecules lacking the handle cannot attach to the solid. The molecules with the handle will stay and those molecules without the affinity handle can be removed or taken away by for example a solution. Then the molecule with the affinity handle can be eluted from the solid resulting in a pure or more pure form of the molecule. The na\dot{i}ve library refers to a library where minimal (unintended) or no selection has occurred. Hard randomization is defined as a process that identifies the residues on the polypeptide that allow for a majority of the affinity to the materials that the polypeptide will have its interactions. Soft randomization is defined as a process that identifies the residues on the polypeptide that allow for the minority of the affinity to the materials that the polypeptide will have its interactions. In one embodiment, soft randomization can occur after hard randomization and can be used to optimize the interaction between a polypeptide and the molecule or molecules with which it interacts. An inhibitor is defined as an agent that interferes with or slows a chemical action. A phage is defined as a bacteriophage. A bacteriophage is a virus whose host is a bacteria or bacterium. It is a virus that replicates within a bacterial cell.

TABLE 10
Increases in efficacy of inhibition of FX activation by
phage display evolution of peptides.

| | | FX activation |
|---|---|---|
| Stage of Evolution of the Peptide | Name of Peptide | (*IC₅₀ in nm) |
| (1) Initial lead compound from | A-53-Z | 4400 ± 800 |
| the naïve library | | |
| (2) Consensus sequence from | C-Z | 5.9 ± 0.9 |
| the hard randomization | | |
| (3) Final soft randomization to | A-100-Z | 1.5 ± 0.7 |
| obtain the final consensus sequence | | |
| (4) Cleaved A-100-Z peptide | A-183 | 1.6 ± 1.2 |

*Inhibitor concentration ± standard error

The evolution of the more effective FVIIa inhibitor resulted in a peptide that was more effective in prolonging clotting times in human plasma.

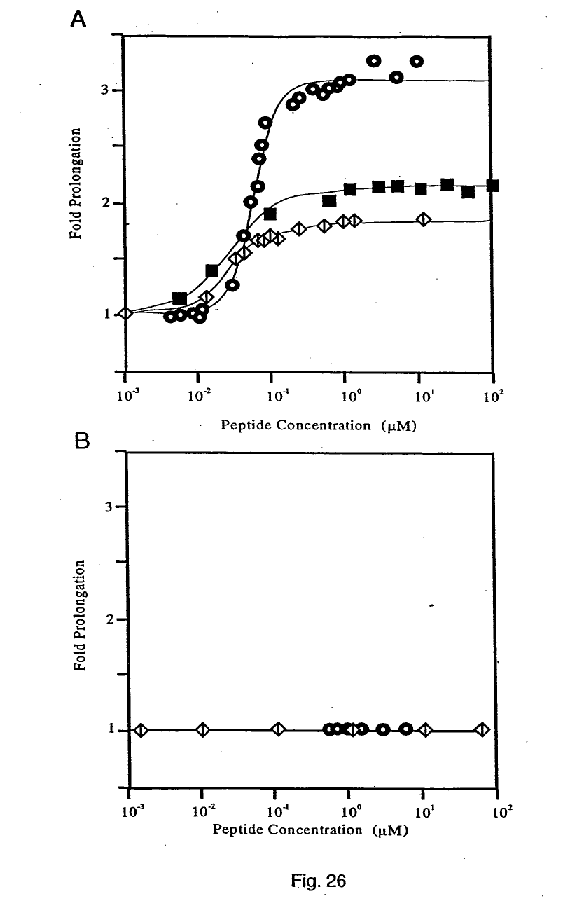

FIG. 26 illustrates the effect of A-series peptides on the prothrombin time (PT) and activated partial thromboplastin time (APTT) in human plasma. The fold prolongation of the clotting times upon (a) initiation by Innovin (human replicated TF and Ca²⁺) in the PT assay or (B) initiation by Actin FS in the APTT assay are plotted versus the concentration of A-183 (□), A-100 (□), and A-100-Z (□□). Uninhibited clotting times were 9.6 and 30.3 s for the PT and APTT, respectively.

FIG. 26 illustrates the effect of A-series peptides on the prothrombin time (PT) and activated partial thromboplastin time (APTT) in human plasma (Dennis et al. 2001). In the upper panel the fold prolongation of the clotting times upon initiation by TF and Ca2+ is given. TF is the specific target site that the proteins have been selected to interact with in solution. In the lower panel initiation by actin FS in the APTT assay. The polypeptides had not been selected to interact with concentrations of the selected peptides: A-183 (closed circle), A-100 (closed box), and A-100-z (open box). The upper panel shows that polypeptides that have been developed by selection can be more effective in prolonging clotting time. In the lower panel neither A-183 nor A-100-z prolonged clotting times. This showed that A-183 and A-100-z were specifically interacting with the TF, which they had been selected with as a target site. (Dennis, M. S., Roberge, M., Quan, C. and R. A. Lazarus. 2001. Selection and characterization of a new class of peptide exosite inhibitors of coagulation factor VIIa. Biochemistry. 40: 9513-9521).

The results of this example illustrate that a (1) lead peptide or protein (or the binding part of the protein) or (2) a na\dot{i}ve peptide library can be matured to identify amino acids, peptides or proteins capable of interacting with a target protein in a much more effective manner.

In one embodiment, the above described technology is utilized to identify and mature peptides, proteins, or selected regions of proteins, where these amino acid sequences are more effective at interacting with proteins involved in conferring resistance to other biocides. In other words, this technique is used to identify and mature toxins (or portions of toxins) that are ultimately more effective in binding with target-sites involved in conferring resistance to other biocides. The phage display technology as described above is an exemplary tool that is used to (1) identify, (2) mature, or (3) identify and mature negative cross-resistance toxins. Phage-display technology has already been used to identify key epitopes in the toxin-receptor interactions in Bt-susceptible Maduca sexta cadherin-like receptors. In addition, phage-display technology can be adapted to determine new target-sites of the cadherin-like receptor useful in development of negative-cross resistance toxins. The toxins may interact with a mutated, truncated, non-glycosolated, or any combination of theses, cadherin-like or cadherin receptor.

Identification and/or maturation of negative cross-resistance toxins occurs, in one embodiment, by selection of the whole toxin or regions of the toxin involved in binding. If a polypeptide sequence was identified with increased binding to the resistant form of the plant, insect, nematode, or bacterial target gene, then the consensus sequence developed from this technology could be engineered into for example, the binding domain of the wild-type toxin. In a further embodiment, this consensus sequence is used in a chimera, where the amino acids are attached to another sequence of amino acids that may directly perform the toxic function in the cell. Thus the evolution of an NCR toxin can be by evolving a toxin directly or evolving a polypeptide sequence that can be used in a toxin that will result in NCR activity of the toxin.

In one embodiment, the above described process is utilized with a toxin that has low toxicity against the resistant organisms as compared to the susceptible organisms. The toxin is then developed by evolving the toxin or a polypeptide sequence (or a single amino acid change) such that the toxin becomes more toxic to the resistant form than what occurred in the original form of the toxin. The evolution of the toxin to being more toxic to the resistant form may be as little as one amino acid change in the protein, to polypeptide fragments of the toxin altered, upwards to the whole polypeptide or protein toxin being altered for greater toxicity to the resistant target site or organism or both.

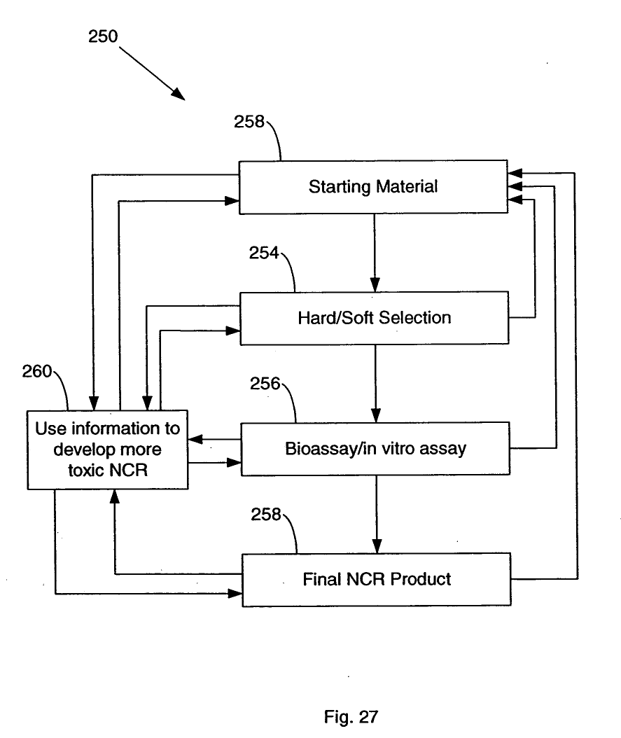

FIG. 27 illustrates a flow chart 250 for evolution of a NCR toxin. A starting material is selected 252. In one embodiment, the starting material is one of a weak, moderate and strong NCR toxin. In an alternative embodiment, the starting material is obtained from bioinformatics-based information. In a further embodiment, the starting material is a naive material, toxins no longer toxic to resistant organisms, and sub-groups thereof. In one embodiment, the starting material is a protein coded by the resistance allele. The resistance allele is an allele from the resistant organism where the allele is involved in conferring the resistance trait to the toxin. The allele may also be variations of this allele either naturally occurring products of mutagensis in either an in vivo or in vitro system. The protein or polypeptide product from the resistant allele of the gene is used in a phage display protocol against naive protein variants or specifically designed or naturally occurring proteins and their variants.

The starting material is then subjected to “hard” and/or “soft” selection 254 against a resistant protein or protein variants that will allow one to select for NCR toxins. Either “hard” or “soft” selection can be used in either order or in any combination to obtain a toxin with greater affinity or toxicity to the resistant target site. A strategy may also be utilized to identify reduced affinity to the susceptible target site instead of the above technique or in combination with any of the approaches given above. At least one of a bioassay and an in vitro assay are utilized 256 to determine increased toxicity to resistance and/or susceptible target sites or organisms. Toxicity, binding affinities, or some other measure can also be determined that indicates the selected polypeptides or proteins that would assist or be directly involved in increased capacity of a toxin to kill resistant lines of the organism. The toxin that results from this selection may also have increased toxicity to the susceptible organisms. Such assays may include, but are not limited to, determining increased mortality rate& or other altered life-history parameters of the resistant organism due to treatment with the evolved (selected) toxin or toxin containing an evolved (selected) component.

A final NCR product is then obtained 258 and is utilized to control a pest population. Such utilization includes development of the NCR product directly. In addition, information gained from the previous steps is used to design components of an NCR compound or polypeptide or protein. Also materials obtained can be utilized to further select NCR compounds by using any polypeptide sequence identified with greater binding to resistant forms in the design of NCR toxins.

In addition, any one of steps 252, 254, 256, and 258 can feed into a second generation of selection that starts with a NCR toxin having at least a slightly increased toxicity. Further, all of steps 252, 254, 256, and 258 can be used in various orders or combinations or at the exclusion of some of the steps. In one embodiment, the final NCR products are deployed in one of three general methods, as described above. The NCR toxin and other compound are deployed together, the NCR toxin and other compound are alternated in time or space or both in the field, and the NCR toxin is deployed in an active refuge model.

Business Models for the Marketing of NCR Products

Negative cross-resistant biocides can be used in a business model to prolong the useful life of a pesticide outside of the normal patent lifetime. A business model is defined as an approach taken to maximize an ability to compete in a market. For example, pesticide or antibiotic can be marketed by an entity for upwards of the twentieth year of the patent. At this point the patent will expire.

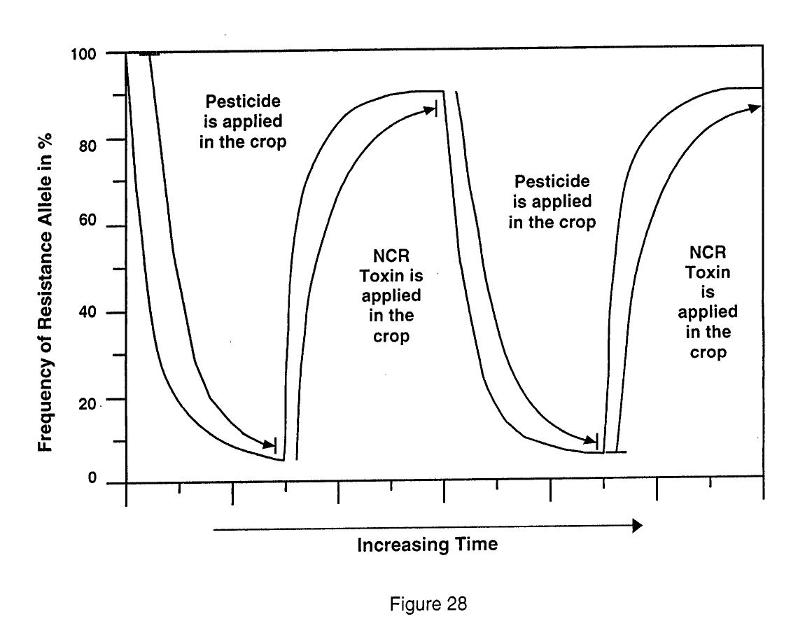

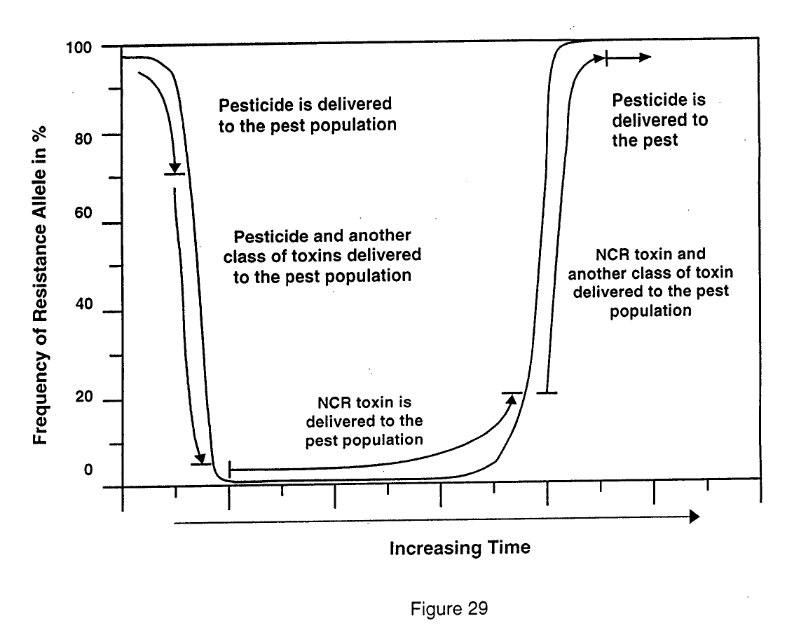

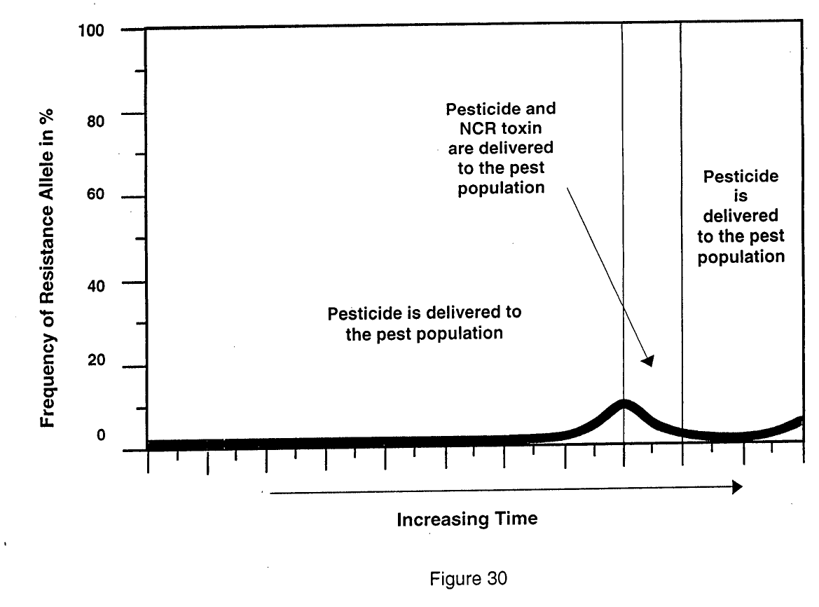

In one embodiment, a entity develops a negative cross-resistant compound prior to or after the expiration of the patent of a pesticide or antibiotic that they manufacture or market. In one example, the company can rotate selling the two compounds (FIGS. 28 and 29). In an alternative embodiment, the entity such as a company sells the NCR compound along with the original pesticide or antibiotic. One example of this is in FIG. 30 illustrates one example in which the two compounds are delivered together to minimize resistance. The two are delivered together infrequently or constantly. The patent on the NCR biocide lasts upwards of twenty years. The development of the first pesticide or antibiotic with an NCR compound provides an advantage to the entity in the market place, since the NCR bioside is less likely to have resistance develop to it than a competitor that does not have the NCR compounds associated with the pesticide or antibiotic. The first entity is able to obtain up to 20 years of protection for the NCR biocide even if the patent as the pesticide or antibiotic has already expired. Thus the NCR biocide adds value to the entity ever after the pesticide or antibiotic may no longer add value to the entity.

Since resistance is likely to emerge near the end of the lifetime of the first biocide's patent, government or regulatory mandates can be put in place requiring the use of NCR products for continued use of the pesticide or antibiotic after a given number of years of use of the first product in the field. Thus, an outside entity could not sell the first biocide, in the absence of a (NCR) second biocide. The development of a third biocide, necessary for the minimization of a dually resistant allele (an allele that is resistant to both the first biocide and the NCR biocide) could in turn add another twenty years to the useful life-time of the first product, bringing the potential total years of useful life of the product to sixty years. This could be continually repeated, where new biocides against alleles could be identified.

A the end of the patent life of the pesticide or antibiotic the NCR product could be sold by a entity as a marketing strategy to suggest to consumers that its product has a practical advantage over a competitors product.

In another embodiment a second entity can manufacture the first entities pesticide or antibiotic when the patent of the first entity has expired. The second entity can also market this pesticide or antibiotic with a NCR product. This would allow the second entity to present its product in the market place as having an advantage of the product of the first entity.

In another embodiment a second entity can manufacture or sell, or both, a NCR product that would work for resistant strains for the first entities pesticide or antibiotic while the patents for the pesticide or antibiotic are still in effect. This allows the second entity to sell a NCR product into the same market niche as the first entity. When and where the first entity sells its pesticide or antibiotic the second entity could sell a NCR product to minimize resistance to the first entities product.

In another embodiment, some pesticides may not be patented or their patents may have already expired. Even if the pesticide is very useful, the lack of exclusivivity means there is little incentive for entities to spend the money to take the product through the regulatory process and market it profitably. An entity could develop a NCR compound useful in deployment with this un-patented or patent expired pesticide to minimize resistance to the un-patented or patent expired pesticide. The patent on the NCR compound allows the entity patent protection for the pesticide plus the NCR product. Other entities would be at a market disadvantage if they marketed and sold only the un-patented or patent expired pesticide.

While the invention has been described in terms of various specific embodiments, those skilled in the art will recognize that the invention can be practiced with modification within the spirit and scope of the claims.

References:

Dennis, M. S., Roberge, M., Quan, C. and R. A. Lazarus. 2001. Selection and characterization of a new class of peptide exosite inhibitors of coagulation factor VIIa. Biochemistry. 40: 9513-9521.

Gómez, I., Oltean, D. I., Gill, S. S., Bravo, A., and M. Soberón. 2001. Mapping the epitope in cadherin-like receptors involved in Bacillus thuringiensis Cry1A toxin interaction using phage. The Journal of Biological Chemistry. 276(31): 28906-28912.

Kiowa, H., Shade, R. E., Zhu-Salzman, K., Subramanian, L., Murdock, L. L., Nielson, S. S., Bressan, R. A., and P. M. Hasegawa. 1998. Phage display selection can differentiate insecticidal activity of soybean cystatins. The Plant Journal. 14(3): 371-379.

1. A method of evaluating the efficacy of a molecule against a target population, the target population including a pest strain resistant to a first toxin, said method comprising:
determining a susceptible pest strain, the susceptible strain being susceptible to the first toxin;
selecting for the resistant strain, the resistant strain being resistant to the first toxin; and
evaluating the efficacy of the resistant strain with a plurality of molecules to determine a second toxin that is more toxic to the resistant strain than to the susceptible strain;
wherein the resistant and susceptible pest strains coexist in the target population.

2. A method in accordance with claim 1 wherein the target population is an insect population.

3. A method in accordance with claim 1 wherein the plurality of molecules are evaluated against only the resistant strain.

4. A method in accordance with claim 1 wherein the plurality of molecules are evaluated against the resistant strain and a subset of molecules are evaluated against the susceptible strains.

5. A method in accordance with claim 1 wherein the plurality of molecules are evaluated against only the heterozygous resistant strain.

6. A method in accordance with claim 1 wherein the plurality of molecules are evaluated against the heterozygous resistant strain and homozygous susceptible strain.

7. A method in accordance with claim 1 wherein the plurality of molecules are evaluated against the heterozygous resistant strain and homozygous resistant strain.

8. A method in accordance with claim 1 wherein the target population is at least one of a mammalian population, a plant population, an animal population, and a virus population.

9. A method in accordance with claim 1 wherein said method further comprises assigning a priority rating to the second toxin if applications of the first toxin and the second toxin are at least as toxic to the bacterial strain containing at least one copy of the resistance gene as to the susceptible bacterial strain.

10. A method for controlling a host species that includes a bacteria existing in a symbiotic relationship with the host species, said method comprising:
determining a susceptible strain of the bacteria that is susceptible to a first toxin;
determining a resistant strain of the bacteria that is resistant to the first toxin;
determining a second toxin that is more toxic to the resistant strain than to the susceptible strain; and
applying the first toxin and the second toxin to the host species such that the host species is adversely impacted.

11. A method in accordance with claim 11 wherein the host species is an insect population.

12. A method in accordance with claim 10 wherein the host species is a mammalian population.

13. A method in accordance with claim 10 wherein application of the first and second toxin has an adverse impact on the bacteria.

14. A method for generating a resistant organism to be used in developing NCR toxins, said method comprising creating genomic changes in the organism to create a resistance trait, wherein the resistant organism is generated for the purpose of developing NCR toxins.

15. A method in accordance with claim 14 further comprising generating a first resistance allele to a first toxin.

16. A method in accordance with claim 15 wherein the resistance allele to the first toxin is generated through at least one of creation of point mutations in genes, genomic changes that involve up-regulation or down regulation of existing genes, mutations that result in a loss of function of the target gene, and genomic changes that cause duplication of genes that result in a resistant trait that will ultimately be used in the discovery of NCR toxins.

17. A method in accordance with claim 14 further comprising generating a dually resistant allele.

18. A method in accordance with claim 17 wherein the dually resistant allele is generated through at least one of creation of point mutations in genes, genomic changes that involve up-regulation or down regulation of existing genes, and genomic changes that cause duplication of genes that result in a resistant trait that will ultimately be used in the discovery of further NCR toxins.

19. A method in accordance with claim 14 wherein the genomic changes occur through at least one of EMS, X-rays, Gamma-rays, radioactive materials, P-elements, mobile genetic elements, ‘jumping genes’, and other genomic technology that result in genomic changes in an organism.

20. A method in accordance with claim 14 wherein the resistant organism is at least one of a naturally occurring insect population, a naturally occurring plant population, a naturally occurring fungi population, a naturally occurring bacterial population, a maintained field, a maintained laboratory, a maintained freezer, and a stock of an organism.

21. A method in accordance with claim 15 further comprising delivering, either externally or internally, to the organism the toxins to be used for selection of the first resistance allele.

22. A method in accordance with claim 17 further comprising delivering, either externally or internally, to the organism the materials to be used for selection of the dually-resistant allele.

23. A method in accordance with claim 14 wherein the resistant organism makes its own toxin.

24. A method in accordance with claim 14 further comprising feeding a toxin to the organism.

25. A method in accordance with claim 14 further comprising delivering the toxin to the organism through at least one of an external membrane and a surface of the organism.

26. A method in accordance with claim 25 further comprising delivering the toxin to the organism using at least one of an organic solvent and an inorganic solvent.

27. A method in accordance with claim 14 wherein the organism produces the toxin internally through transgenic means.

28. A method in accordance with claim 14 wherein the toxin is delivered internally to the target organism by injection.

29. A method in accordance with claim 14 wherein the toxin is delivered by organisms that are symbiotically associated with the resistant organism.

30. A method in accordance with claim 29 wherein the delivery organisms are transgenic organisms.

31. A method in accordance with claim 14 wherein the toxin is delivered by organisms that live in association with the target organism.

32. A method in accordance with claim 14 wherein the target genes for the toxins include, but are not limited to, Cytochrome P450 enzymes, Esterase enzymes, ion pumps, glutathione-S-transferases, voltage-gated sodium channels, sodium channels, calcium channels, membrane transport proteins, GABA receptors, GABA-gated chloride channel, nicotinic acetylcholine receptors, calcium channels, amino-peptidases, proteases, a-amylases, lipases, and allelic variants.

33. A method in accordance with claim 14 wherein the toxins used to select the resistance trait include, but are not limited to, target genes in insects of the Bacillus thuringiensis insecticidal toxins, target genes in insects of the Photorhabdus luminescens insecticidal toxins, target genes in insects of the Xenorhabdus nematophilus insecticidal toxins, target genes in insects of Spinosad, target genes in insects of Spinosyn, target genes in insects of imidacloprid, and allelic variants.

34. A method in accordance with claim 14 wherein the toxins used to select the resistance trait include, but are not limited to, DDT, pyrethroids, AaIT scorpion toxin, chlorinated hydrocarbons, organophosphates, and carbamates.

35. A method in accordance with claim 14 wherein a target site for the development of NCR toxins is at least one gene that metabolizes a first toxin.

36. A method in accordance with claim 14 wherein the resistant organism is associated with resistance to toxins through at least one of reduced penetration by the toxin and resistance to toxins through sequestration of the toxin.

37. A method in accordance with claim 15 wherein the resistant allele is obtained in a screen for allelic variants of a gene causing resistance using an in vitro system.

38. A method in accordance with claim 17 wherein the dually resistant allele is obtained in a screen for allelic variants of a gene causing resistance using an in vitro system.

39. A method of screening for negative cross resistance with respect to a target allele, said method comprising:
evaluating whether a first toxin that was originally effective against a susceptible line of organism is effective against a susceptible line of organism; and
if the toxin is no longer effective against the susceptible line of organism, evaluating whether the toxin is effective against a resistant line of organism.

40. A method in accordance with claim 39 wherein the toxin is evaluated against a second line of organism homozygous for the target allele to identify high levels of toxicity.

41. A method in accordance with claim 39 wherein the first toxin is evaluated against organisms heterozygous for second alleles considered to be resistant to the first toxin and susceptible to a second toxin and wherein the second allele is resistant to the second toxin and is susceptible to the first toxin.

42. A method in accordance with claim 39 wherein homozygous insects that are resistant to a second toxin are evaluated to be susceptible to the first toxin.

43. A method in accordance with claim 39 further comprising delivering the first toxin to an external surface of the organism.

44. A method in accordance with claim 39 wherein the toxin is delivered using at least one of an organic solvent and an inorganic solvent.

45. A method in accordance with claim 39 further comprising feeding the toxin to the organism.

46. A method in accordance with claim 39 further comprising delivering the toxin by organisms that are symbiotically associated with the susceptible organism.

47. A method in accordance with claim 39 further comprising delivering the toxin by transgenic organisms that are symbiotically associated with the susceptible organism.

48. A method in accordance with claim 39 further comprising delivering toxin internally to the organism using at least one of an inorganic solvent, an organic solvent, transgenic techniques, transgenic organisms, P-element transformation of the target organism with the toxin, mobile genetic elements, agrobacterium transformation of the target organism with the toxin, and particle gun transformation of the target organism with the toxin.

49. A method in accordance with claim 39 wherein the toxin is a second-generation insecticide.

50. A method in accordance with claim 39 wherein the toxin includes at least one of Bacillus thuringiensis insecticidal toxins, Photorhabdus luminescens insecticidal toxins, Xenorhabdus nematophilus insecticidal toxins, Spinosad, Spinosyn, imidacloprid and derivatives thereof.

51. A method in accordance with claim 39 wherein the toxin includes at least one of DDT, pyrethroids, AaIT scorpion toxin, chlorinated hydrocarbons, organophosphates, and carbamates.

52. A method in accordance with claim 50 wherein the Bacillus thuringiensis insecticidal toxin is altered such that it is no longer toxic to susceptible lines of the insects and the toxin is subsequently used in a bioassay to determine that the toxin kills the resistant organisms.

53. A method in accordance with claim 50 wherein the Photorhabdus luminescens insecticidal toxin is subsequently used in a bioassay to determine that the toxin kills the resistant organisms.

54. A method in accordance with claim 50 wherein the Xenorhabdus nematophilus insecticidal toxin is altered such that it is no longer toxic to susceptible lines of insect and is subsequently evaluated to determine that the toxin kills the resistant insects.

55. A method in accordance with claim 50 wherein the Spinosyn or Spinosad compounds are altered such that they are no longer toxic to the susceptible line of organisms and are subsequently used in a bioassay to determine that the toxin kills the resistant organisms.

56. A method in accordance with claim 50 wherein the imidicloprid compounds are altered such that they are no longer toxic to susceptible lines of organisms and are subsequently used in a bioassay to determine that the toxin kills the resistant organisms.

57. A method in accordance with claim 50 wherein the AaIT scorpion toxins are altered such that they are no longer toxic to susceptible lines of the organism and are subsequently used in a bioassay to determine that the toxin kills the resistant organisms.

58. A method in accordance with claim 51 wherein second generation toxins are altered such that they are no longer toxic to susceptible lines of the organism and the toxins are subsequently used in a bioassay to determine that the toxin kills the resistant organisms.

59. A method in accordance with claim 39 wherein the toxins are altered by changing coding sequence in a toxin gene.

60. A method of screening compounds for negative cross resistance activity comprising:
altering compounds originally toxic to susceptible insects such that they are no longer toxic to susceptible lines of insects; and
using the altered compounds in a bioassay to determine whether the toxin will kill resistant organisms.

61 A method of evaluating the efficacy of a molecule against a target population, the target population including a strain resistant to a first toxin, said method comprising:
determining a susceptible strain in the target population, the susceptible strain being susceptible to the first toxin;
selecting for the resistant strain in the target population, the resistant strain being resistant to the first toxin; and
evaluating the efficacy of the resistant strain with a plurality of molecules to determine a second toxin that is more toxic to the resistant strain than to the susceptible strain;

62. A method in accordance with claim 61 wherein the target population is at least one of a fungi, a plant, and a nematode.

63. A method in accordance with claim 61 further comprising:
evaluating the efficacy of a heterozygous strain of the target population with separate applications of the first toxin and the second toxin; and
assigning a priority rating to the second toxin if the separate applications of the first toxin and the second toxin are at least as toxic to the heterozygous strain as to the susceptible strain.

64. A method in accordance with claim 61 further comprising screening the heterozygous strain with the first toxin and the second toxin applied at the same time.

65. A method in accordance with claim 63 further comprising assigning a priority rating to the second toxin if the application of the first toxin and the second toxin at the same or substantially the same time are at least as toxic to the heterozygous strain as to the susceptible strain.

66. A method in accordance with claim 61 further comprising determining whether both the first toxin and the second toxin can be applied to the target population at the same or substantially the same time at an acceptable rate.

67. A method in accordance with claim 66 further comprising assigning a priority rating to the second toxin if the first toxin and the second toxin can be applied to the target population at the same or substantially the same time at an acceptable rate.

68. A method in accordance with claim 61 wherein selecting for the resistant strain in the target population comprises selecting for a homozygous resistant strain in the target population.

69. A method in accordance with claim 61 wherein selecting for the resistant strain in the target population comprises selecting for a resistant strain in the target population using at least one of a field collected line and an EMS-mutagenized line.

70. A method in accordance with claim 61 wherein evaluating the efficacy of the resistant strain comprises evaluating the efficacy of the resistant strain with between about 10 and 10⁹ molecules.

71. A method of testing for negative cross resistance in a target population, said method comprising:
determining a susceptible strain (S/S) in the target population, the susceptible strain (S/S) susceptible to a first toxin;
selecting for a resistant strain (R/R) in the target population, the resistant strain (R/R) resistant to the first toxin; and
evaluating the efficacy of the resistant strain (R/R) with between about 10 and 10⁹ molecules to determine a second toxin that is more toxic to the resistant strain (R/R) than to the susceptible strain (S/S);

72. A method in accordance with claim 71 wherein the target population is at least one of a fungi, a plant, and a nematode.

73. A method in accordance with claim 71 further comprising:
evaluating the efficacy of a heterozygous strain (R/S) of the target population with separate applications of the first toxin and the second toxin to determine if separate applications of the first toxin and the second toxin are at least as toxic to the heterozygous strain (R/S) as to the susceptible strain (S/S); and
assigning a high negative cross resistance priority to the second toxin if the separate applications of the first toxin and the second toxin are at least as toxic to the heterozygous strain (R/S) as to the susceptible strain (S/S).

74. A method in accordance with claim 71 further comprising evaluating the efficacy of the heterozygous strain (R/S) with the first toxin and the second toxin applied at the same or substantially the same time to determine if the application of the first toxin and the second toxin at the same or substantially the same time is at least as toxic to the heterozygous strain (R/S) as to the susceptible strain (S/S).

75. A method in accordance with claim 73 further comprising determining whether both the first toxin and the second toxin can be applied to the target population at the same or substantially the same time at an economically acceptable rate.

76. A method in accordance with claim 74 further comprising assigning a high negative cross resistance priority to the second toxin if the first toxin and the second toxin can be applied to the target population at the same or substantially the same time at an economically acceptable rate.

77. A method in accordance with claim 71 wherein selecting for a resistant strain (R/R) in the target population comprises selecting for a resistant strain (R/R) in the target population using at least one of a field collected line and an EMS-mutagenized line.

78. A method of using a first negative cross-resistance (NCR) toxin and a second NCR toxin against a pest population in a refuge to selectively kill heterozygotes and homozygotes carrying resistance alleles to the first NCR toxin, wherein the first NCR toxin is used in a main field and the second NCR toxin is used in the refuge.

79. A method in accordance with claim 78 wherein the second toxin comprises at least one of a Bacillus thuringiensis protein toxin, a lectin protein toxin, a Saccharopolyspora spinosa protein toxin, a Photorhabdus luminescens protein toxin, a Xenorhabdus nematophilus protein toxin, a imidacloprid toxin, a Cysteine protease inhibitors protein toxin, a Bowman-Birk Inhibitors protein toxin, a Kunitz inhibitors protein toxin, and an alpha-amylase inhibitor protein toxin.

80. A method in accordance with claim 78 wherein a target site for the second NCR toxin is at least one of a Cadherin gene protein and a truncated Cadherin gene protein.

81. A method in accordance with claim 78 further comprising using the second toxin to slow the rate at which resistance enters the pest population in the field.

82. A method in accordance with claim 78 further comprising using the second toxin to slow the rate at which the resistance allele enters the pest population in the field.

83. A method in accordance with claim 78 further comprising using the second toxin to decrease the level of resistance in the pest population in the field.

84. A method in accordance with claim 78 further comprising using the second toxin to decrease the levels of the resistance allele in the pest population in the field.

85. A method in accordance with claim 78 further comprising using the second toxin to maintain the level of resistance in the pest population in the field.

86. A method in accordance with claim 78 further comprising using the second toxin to maintain the levels of the resistance allele in the pest population in the field.

87. A method in accordance with claim 78 wherein the second toxin is carried by plants in the refuge, the plants containing the second toxin are planted separate from the transgenic plants containing other toxins.

88. A method in accordance with claim 78 wherein the second toxin is carried by plants in the refuge, the plants containing the second toxin are planted within or near the region where the transgenic plants containing other toxins are planted.

89. A method in accordance with claim 78 further comprising delivering the first toxin through a transgenic plant.

90. A method in accordance with claim 78 where the pest population is one of an insect population, a nematode population, a plant population, a fungi population, and a bacterial population.

91. A method in accordance with claim 78 further comprising delivering the second toxin through a transgenic plant.

92. A method in accordance with claim 91 wherein the transgenic plant is the same or substantially the same species as a crop grown in the main field.

93. A method in accordance with claim 91 wherein the transgenic plant is a different species from a crop grown in the main field.

94. A method in accordance with claim 78 further comprising delivering at least one of the first toxin and the second toxin as a spray.

95. A method in accordance with claim 78 further comprising delivering the second toxin in a transgenic plant in the refuge field in as few as 1/10,000,000 of the total plants in the main field.

96. A method in accordance with claim 78 further comprising delivering the second toxin in a transgenic plant in the refuge field that is up to 9,999,999/10,00,000 of the total plants in the main field.

97. A method of using a first negative cross-resistance (NCR) toxin and a second NCR toxin against a pest population in a refuge to selectively kill heterozygotes that carry resistance alleles to the first NCR toxin which is used in the main field.

98. A method in accordance with claim 97 wherein the first toxin is one of a Bacillus thuringiensis protein toxin, a Saccharopolyspora spinosa protein toxin, a Photorhabdus luminescens protein toxin, a Xenorhabdus nematophilus protein toxin, a imidacloprid toxin, a lectin protein toxin, a Cysteine protease inhibitors protein toxin, a Bowman-Birk Inhibitors protein toxin, a Kunitz inhibitors protein toxin, and a 1 alpha-amylase inhibitor protein toxin.

99. A method in accordance with claim 97 further comprising delivering at least one of the first toxin and the second toxin in a transgenic plant.

100. A method in accordance with claim 97 where the target site for the first NCR toxin is at least one of a Cadherin gene and pseudogene.

101. A method in accordance with claim 97 wherein the target population is one of an insect population, a nematode population, a plant population, a fungi population, and a bacterial population.

102. A method in accordance with claim 97 further comprising delivering the second toxin through a plant that is the same or substantially the same species as a crop grown in a main field.

103. A method in accordance with claim 97 further comprising delivering the second toxin through a plant that is a different species from a crop grown in a main field.

104. A method in accordance with claim 97 further comprising delivering at least one of the first toxin and the second toxin as a spray.

105. A method in accordance with claim 97 further comprising delivering the second toxin in a transgenic plant in the refuge field in as few as 1/10,000,000 of the total plants in the main field.

106. A method in accordance With claim 97 further comprising delivering the second toxin in a transgenic plant in the refuge field that is up to 9,999,999/10,00,000 of the total plants in the field.

107. A method in accordance with claim 97 further comprising planting refuge plants containing the NCR toxin separate from the transgenic plants containing other toxins.

108. A method in accordance with claim 97 further comprising planting refuge plants containing the NCR toxin within the region where transgenic plants containing other toxins are planted.

109. A method in accordance with claim 97 further comprising utilizing the second toxin to slow the rate at which resistance enters the pest population in the main field.

110. A method in accordance with claim 97 further comprising utilizing the second toxin to slow the rate at which the resistance allele enters the pest population in the main field.

111. A method in accordance with claim 97 further comprising utilizing the second toxin to decrease the level of resistance in the pest population in the main field.

112. A method in accordance with claim 97 further comprising utilizing the second toxin to decrease the levels of the resistance allele in the pest population in the main field.

113. A method in accordance with claim 97 further comprising utilizing the second toxin to maintain the level of resistance in the pest population in the main field.

114. A method in accordance with claim 97 further comprising utilizing the second toxin to maintain the levels of the resistance allele in the pest population in the main field.

115. A method of evaluating the efficacy of a molecule against a target population, the target population including a strain resistant to a Bacillus thuringiensis insecticidal toxin, said method comprising:
determining a susceptible strain susceptible to a Bacillus thuringiensis insecticidal toxin;
selecting for the resistant strain, the resistant strain being resistant to the Bacillus thuringiensis insecticidal toxin; and
evaluating the efficacy of the resistant strain with a plurality of molecules to determine a second toxin that is more toxic to the resistant strain than to the susceptible strain.

116. A method in accordance with claim 115 wherein the target population is an insect population.

117. A method in accordance with claim 115 wherein the target population is a nematode population.

118. A method in accordance with claim 115 further comprising assigning a priority rating to the second toxin if applications of the Bacillus thuringiensis insecticidal toxin and the second toxin are at least as toxic to the heterozygous insect strain as to the susceptible bacterial strain.

119. A method in accordance with claim 115 further comprising assigning a priority rating to the second toxin if applications of the Bacillus thuringiensis insecticidal toxin and the second toxin are at least as toxic to the heterozygous nematode strain as to the susceptible bacterial strain.

120. A method in accordance with claim 115 wherein the negative cross-resistance toxin targets a cadherin gene protein in insects.

121. A method in accordance with claim 115 wherein the negative cross-resistance toxin targets a truncated cadherin gene protein in insects.

122. A method in accordance with claim 115 wherein the negative cross-resistance toxin targets a non-glycosolated cadherin gene protein in insects.

123. A method in accordance with claim 115 wherein the negative cross-resistance toxin targets a glycosolated cadherin gene protein in insects.

124. A method in accordance with claim 115 wherein the negative cross-resistance toxin targets the cadherin gene protein in Heliothis virescens.

125. A method in accordance with claim 115 wherein the negative cross-resistance toxin targets a non-functional cadherin gene protein in Heliothis virescens.

126. A method in accordance with claim 115 wherein the negative cross-resistance toxin targets a truncated cadherin gene protein in Heliothis virescens.

127. A method in accordance with claim 115 wherein the negative cross-resistance toxin targets the r1 Bacillus thuringiensis allele in Heliothis virescens.

128. A method in accordance with claim 115 wherein the negative cross-resistance toxin targets the beta-1,3-galactosyltransferase gene in Heliothis virescens.

129. A method in accordance with claim 115 wherein the negative cross-resistance toxin targets a cadherin gene protein in nematodes.

130. A method in accordance with claim 115 wherein the negative cross-resistance toxin targets a truncated cadherin gene protein in nematodes.

131. A method in accordance with claim 115 wherein the negative cross-resistance toxin targets a non-glycosolated cadherin gene protein in nematodes.

132. A method in accordance with claim 115 wherein the negative cross-resistance toxin targets a glycosolated cadherin gene protein in nematodes.

133. A method in accordance with claim 115 wherein the negative cross-resistance toxin targets the cadherin gene protein in nematodes.

134. A method in accordance with claim 115 wherein the negative cross-resistance toxin targets a non-functional cadherin gene protein in nematodes.

135. A method in accordance with claim 115 wherein the negative cross-resistance toxin targets a truncated cadherin gene protein in nematodes.

136. A method in accordance with claim 115 wherein the negative cross-resistance toxin targets the bre-5 Bacillus thuringiensis resistant allele in nematodes.

137. A method in accordance with claim 115 wherein the negative cross-resistance toxin targets the beta-1,3-galactosyltransferase gene in nematodes.

138. A method in accordance with claim 115 wherein the genomic changes are naturally occurring and are selected for in at least one of the laboratory and in the field.

139. A method for evolving a strengthened NCR toxin from an initial NCR toxin, said method comprising the steps of obtaining an initial NCR compound;
selectively increasing the toxicity of the NCR compound; and
testing the evolved compound to determine if the evolved compound is a stronger NCR compound than the initial NCR toxin.

140. A method in accordance with claim 139 further comprising using a laboratory evolved negative cross-resistance toxin in a refuge to selectively kill at least one of heterozygotes and homozygotes carrying resistance alleles to the toxin used in the main field.

141. A method in accordance with claim 139 wherein the toxin used in the main field is the Bacillus thuringiensis protein toxin,.a lectin protein toxin, the Saccharopolyspora spinosa protein toxin and the Photorhabdus luminescens protein toxin.

142. A method in accordance with claim 139 wherein the target site for the NCR toxin is at least one of a Cadherin gene protein and truncated Cadherin gene protein.

143. A method in accordance with claim 139 wherein the toxin used in the refuge is used to slow the rate at which resistance enters the pest population in the field.

144. A method in accordance with claim 139 wherein the toxin used in the refuge is used to slow the rate at which at least one resistance allele enters the pest population in the field.

145. A method in accordance with claim 139 wherein the toxin used in the refuge is used to decrease the level of resistance in the pest population in the field.

146. A method in accordance with claim 139 wherein the toxin used in the refuge is used to decrease the levels of the resistance allele or alleles in the pest population in the field.

147. A method in accordance with claim 139 wherein the toxin used in the refuge is used to maintain the level of resistance in the pest population in the field.

148. A method in accordance with claim 139 wherein the toxin used in the refuge is used to maintain the levels of at least one resistance allele in the pest population in the field.

149. A method in accordance with claim 139 wherein the refuge plants containing the NCR toxin are planted separate from the transgenic plants containing other toxins are planted.

150. A method in accordance with claim 139 wherein the refuge plants containing the NCR toxin are planted within or near the region where the transgenic plants containing other toxins are planted.

151. A method in accordance with claim 139 wherein the toxin used in the main field is delivered in a transgenic plant.

152. A method in accordance with claim 139 wherein the target population is one of an insect population, nematode population, plant population, fungi population, and bacterial population.

153. A method in accordance with claim 139 wherein the NCR toxin used in the refuge field is delivered in a transgenic plant.

154. A method in accordance with claim 139 wherein the NCR toxin used in the refuge field is delivered in a transgenic plant that is the same or substantially the same species as the crop grown in the main field.

155. A method in accordance with claim 139 wherein the NCR toxin used in the refuge field is delivered in a transgenic plant that is a different species from the crop grown in the main field.

156. A method in accordance with claim 139 wherein the toxin used in the main field is delivered as a spray.

157. A method in accordance with claim 139 wherein the toxin is delivered to an organism through at least one of a vaccine, a pill, a gel tablet, an injection, a syrup, a powder, or mixed in a food or drink product.

158. A method in accordance with claim 139 wherein the NCR 5 toxin used in the refuge field is delivered as a spray.

159. A method in accordance with claim 139 wherein the NCR toxin used in the refuge field is delivered in a transgenic plant in the field that is as few as 0.00001 percent of the total plants in the field.

160. A method in accordance with claim 139 wherein the NCR toxin used in the refuge field is delivered in a transgenic plant in the field that is up to 99.99999 percent of the total plants in the field.

161. A method in accordance with claim 139 wherein the NCR toxin used in the refuge field is delivered in a transgenic plant in the field is as few as 0.00001 percent or is up to 99.99999 percent or inclusive of these two extremes of the total plants in the field.

162. A method in accordance with claim 70 further comprising delivering the second toxin in a transgenic plant in the refuge field that is up to 99,999/100,000 of the total plants in the main field.

163. A method in accordance with claim 70 further comprising delivering the second toxin in a transgenic plant in the refuge field in as few as 1/10,000 of the total plants in the main field.

164. A method in accordance with claim 70 further comprising delivering the second toxin in a transgenic plant in the refuge field that is up to 9,999/10,000 of the total plants in the main field.

165. A method in accordance with claim 70 further comprising delivering the second toxin in a transgenic plant in the refuge field in as few as 1/1,000 of the total plants in the main field.

166. A method in accordance with claim 70 further comprising delivering the second toxin in a transgenic plant in the refuge field that is up to 999/10,000 of the total plants in the main field.
